(19) United States
(12) Patent Application Publication
(10) Pub. No.: US 2025/0275853 A1
(43) Pub. Date: Sep. 4, 2025

(54) LEAFLET CLAMPING MEMBERS

(71) Applicant: EDWARDS LIFESCIENCES CORPORATION, Irvine, CA (US)

(72) Inventors: Noam Nir, Pardes-Hanna (IL); Michael Bukin, Pardes Hanna (IL); Eran Goldberg, Nesher (IL); Noa Axelrod Manela, Kadima (IL); Elena Sherman, Pardes Hana (IL)

(73) Assignee: EDWARDS LIFESCIENCES CORPORATION, Irvine, CA (US)

(21) Appl. No.: 19/212,454

(22) Filed: May 19, 2025

Related U.S. Application Data
(63) Continuation of application No. PCT/US2023/082574, filed on Dec. 5, 2023.
(60) Provisional application No. 63/387,909, filed on Dec. 16, 2022.

(51) Int. Cl. A61F 2/24

(52) U.S. Cl. CPC A61F 2/2418; A61F 2220/0066; A61F 2240/001

(57) ABSTRACT

The present disclosure relates to prosthetic valve that include leaflets coupled to a frame by clamping members, optionally in a sutureless manner. In an example, a prosthetic valve can include at least one stapling member coupling at least one leaflet of a plurality of leaflets to the frame, wherein the clamping member comprises a plurality of stapling portions separated from each other along a spine of the clamping member. Each stapling member can include a pair of legs extending from opposite ends of a backspan, wherein the legs extend through the leaflet and are bent around a corresponding strut of the frame, thereby coupling the leaflet to the frame.

### CROSS-REFERENCE TO RELATED APPLICATIONS

This application is a continuation of PCT Patent Application No. PCT/US2023/082574, filed Dec. 5, 2023, which claims the benefit of U.S. Provisional Application No. 63/387,909, filed Dec. 16, 2022, each of which is incorporated by reference in its entirety herein.

### FIELD

The present invention relates to prosthetic valves, and in particular, to prosthetic valves that include leaflets coupled to frames thereof by clamping members such as stapling members, compressible inserts, or gripping tubes.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Conventional surgically implantable prosthetic valves typically include a leaflet assembly mounted within a relatively rigid support frame or ring. Components of the prosthetic valve are usually assembled with one or more biocompatible fabrics, and a fabric-covered sewing ring is provided around the valve for suturing to the tissue of the native leaflet.

Since surgeries are prone to an abundance of clinical complications, alternative less invasive techniques of delivering a prosthetic heart valve over a catheter and implanting it over the native malfunctioning valve have been developed over the years. Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery assembly for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery assembly can be retrieved thereafter.

### SUMMARY

Most expandable, prosthetic valves comprise an annular frame and prosthetic leaflets mounted inside the frame. The leaflets can be coupled along cusp edges thereof to the frame, either by being sutured to an inner skirt disposed around an inner surface of the frame, wherein the skirt is sutured, in turn, to the frame, or by the cusp edges being directly sutured to selected struts of the frame. The process of suturing leaflets to the frame, either directly to the struts or via an intermediate inner skirt member, can be relatively labor-intensive and involve relatively long process times, which can also result in increased costs. Accordingly, a need exists for improved leaflet attachment solutions to prosthetic valves, which can reduce process times, as well as the difficulty of the assembly procedure.

According to some aspects of the disclosure, there is provided a prosthetic valve comprising a frame movable between a radially compressed state and a radially expanded state, a valvular structure coupled to the frame and comprising a plurality of leaflets configured to regulate flow through the prosthetic valve, and at least one stapling member coupling at least one of the plurality of leaflets to the frame. The at least one stapling comprises a spine extending along a stapling member axis, and a plurality of stapling portions separated from each other by intermediate portions of the spine. Each stapling portion comprises at least one leg extending from a backspan. The at least one leg of each stapling portion extends through a corresponding one of the plurality of leaflets, and is bent around a corresponding strut of a plurality of struts of the frame, so as to couple the leaflet to the frame.

According to some aspects of the disclosure, there is provided a method of assembling a prosthetic valve comprising providing a stapling member in a first bent configuration, approximating a leaflet to a strut of a frame of a prosthetic valve, pushing the legs toward the leaflet such that tips of the legs pierce through the leaflet, extending the legs radially through the leaflet and across the strut, and transitioning the stapling member to a second bent configuration by bending the legs at second bends thereof over the struts, so as to couple the leaflet to the strut. The stapling member in a first bent configuration comprises a spine extending along a stapling member axis and a plurality of U-shaped stapling portions separated from each other by intermediate portions of the spine, each stapling portion comprising a backspan and a pair of legs bent relative to the backspan at first bends of the legs.

According to some aspects of the disclosure, there is provided a prosthetic valve comprising a frame movable between a radially compressed state and a radially expanded state, a valvular structure coupled to the frame and comprising a plurality of leaflets configured to regulate flow through the prosthetic valve, and at least one compressible insert disposed within a recess formed in at least one of the recessed struts. The frame comprises a plurality of intersecting struts, the plurality of intersecting struts comprising recessed struts and unrecessed struts. The at least one of the plurality of leaflets is coupled to at least one of the recessed struts by having a cusp end portion thereof clamped inside the recess of the corresponding recessed strut by the corresponding at least one compressible insert.

According to some aspects of the disclosure, there is provided a method of assembling a prosthetic valve comprising approximating a cusp end portion of at least one leaflet to an inner surface of a recessed strut of a frame of a prosthetic valve, and pushing a compressible insert, along with a portion of the cusp end portion, into a recess formed in the corresponding recessed strut, thereby coupling the cusp end portion to the frame.

According to some aspects of the disclosure, there is provided a prosthetic valve comprising a frame comprising a base portion and a plurality of vertically oriented commissure posts extending from the base portion, a valvular structure coupled to the frame and comprising a plurality of leaflets configured to regulate flow through the prosthetic valve, and at least one gripping tube. The at least one gripping tube comprises an inner channel and a slot extending between free ends of side arms of the gripping tube. The at least one of the plurality of leaflets is coupled to the frame by the at least one gripping tube disposed around a cusp end portion of the leaflet which is at least partially wrapped around at least a part of the base portion retained within the inner channel.

According to some aspects of the disclosure, there is provided a method of assembling a prosthetic valve comprising positioning a cusp end portion of at least one leaflet between a base portion of a frame of a prosthetic valve and at least one gripping tube, and pushing the gripping tube toward the cusp end portion and the base portion, such that a slot of the gripping tube expands to let the base portion and the cusp end portion slide into an inner channel of the gripping tube, until the base portion resides within the inner channel with the cusp end portion at least partially wrapped around the base portion, at which point side arms defining the slot of the gripping tube are allowed to snap back toward each and clamp against a portion of the leaflet extending through the slot, so as to couple the at least one leaflet to the base portion.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of an exemplary prosthetic valve that includes a leaflet structure coupled to an inner skirt.

FIG. 1B is a perspective view of the prosthetic valve of FIG. 1A, without the outer skirt.

FIG. 1C is a perspective view of a frame of the prosthetic valve of FIGS. 1A-1B.

FIG. 2 shows leaflets of a valvular structure of the prosthetic valve of FIGS. 1A-1B.

FIG. 3 is a perspective view of an exemplary prosthetic valve that includes a leaflet structure sutured to struts of the frame.

FIG. 4 shows an exemplary delivery assembly comprising a delivery apparatus carrying a prosthetic device.

FIG. 5A shows a portion of an exemplary stapling member.

FIG. 5B is a sectional view taken along line 5B-5B of FIG. 5A.

FIG. 5C shows an enlarged view of an end portion of a leg of the stapling member of FIG. 5A.

FIG. 6A shows the stapling member of FIG. 5A in a first bent configuration.

FIG. 6B is a sectional view taken along line 6B-6B of FIG. 6A.

FIG. 7A shows the stapling member of FIG. 5A in a second bent configuration.

FIG. 7B is a sectional view taken along line 7B-7B of FIG. 7A.

FIG. 8A shows a portion of an exemplary stapling member, comprising legs having arrow-shaped tips.

FIG. 8B is a sectional view of a stapling portion, taken along line 8B-8B of FIG. 8A.

FIG. 8C shows the stapling portion of FIG. 8B in a second bent configuration.

FIG. 9A is a sectional view of a stapling portion comprising a stapling element, shown in a first bent configuration.

FIG. 9B shows the stapling portion of FIG. 9A in a second bent configuration.

FIG. 10 shows a leaflet positioned between a strut and a stapling member, prior to being coupled by the stapling member to the strut.

FIGS. 11A-11B show a leaflet sandwiched between the strut and the stapling member in a first bent configuration of the stapling member.

FIG. 12 shows the leaflet coupled to the strut by the stapling member shown in FIGS. 11A-11B, in the second bent configuration of the stapling member.

FIG. 13A shows an exemplary stapling member in a first bent configuration, wherein the legs of each stapling portion are offset from each other.

FIG. 13B shows an enlarged view of one of the stapling portions of FIG. 13A.

FIG. 14A shows the stapling member of FIG. 13A in a second bent configuration.

FIG. 14B shows an enlarged view of one of the stapling portions of FIG. 14A.

FIGS. 15A-15B show a leaflet sandwiched between the strut and a stapling member with offset legs, in a first bent configuration of the stapling member.

FIG. 16 shows the leaflet coupled to the strut by the stapling member shown in FIGS. 15A-15B, in the second bent configuration of the stapling member.

FIG. 17 is a perspective view of an exemplary prosthetic valve that includes a leaflet structure coupled to the frame by stapling members.

FIG. 18 shows an exemplary leaflet comprising a stiff portion.

FIG. 19 shows the leaflet of FIG. 18 coupled to a strut by a stapling member.

FIG. 20A shows an exemplary stapling member formed of a continuous wire, with tips of the legs comprised of lateral bent extensions.

FIG. 20B shows the stapling member of FIG. 20A in a first bent configuration.

FIG. 20C shows the stapling member of FIG. 20A in a second bent configuration.

FIG. 21A shows an exemplary stapling member formed of a continuous wire, with pointed tips of the legs.

FIG. 21B shows the stapling member of FIG. 21A in a first bent configuration.

FIG. 21C shows the stapling member of FIG. 21A in a second bent configuration.

FIG. 22 shows a portion of an exemplary frame comprising recessed struts and unrecessed struts.

FIG. 23 shows a portion of an exemplary frame comprising recessed struts which are wider than unrecessed struts.

FIG. 24 shows an exemplary compressible insert.

FIG. 25A shows a cross-sectional view of a leaflet disposed between a recessed strut and a compressible insert, prior to the leaflet being couple to the strut.

FIG. 25B shows a cross-sectional view of a leaflet coupled to the recessed strut by the compressible insert.

FIG. 26 shows an exemplary surgically implantable prosthetic valve.

FIG. 27 is an exploded view of components of an exemplary prosthetic valve comprising gripping tubes.

FIG. 28 shows a leaflet coupled to a frame of an exemplary prosthetic valve by a gripping tube.

FIG. 29A shows a cross-sectional view of leaflet coupled to a strut by a gripping tube, with the cusp edge retained within the inner channel of the gripping tube.

FIG. 29B shows a cross-sectional view of leaflet coupled to a strut by a gripping tube, with the cusp edge disposed out of the inner channel of the gripping tube.

FIG. 30 shows an exemplary gripping tube that includes gripping tube cusp portions and gripping tube commissure portions.

FIG. 31 shows a portion of an exemplary stapling member comprising blunt tips positioned next to an exemplary leaflet comprising preformed perforations.

FIG. 32A shows a portion of an exemplary stapling member comprising a single leg in its stapling portion, prior to coupling a leaflet to a strut.

FIG. 32B shows the leaflet coupled to the strut by the stapling member of FIG. 32A, in the second bent configuration of the stapling member.

FIG. 33 shows a portion of an exemplary stapling member having a zig-zagged spine.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the elements. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

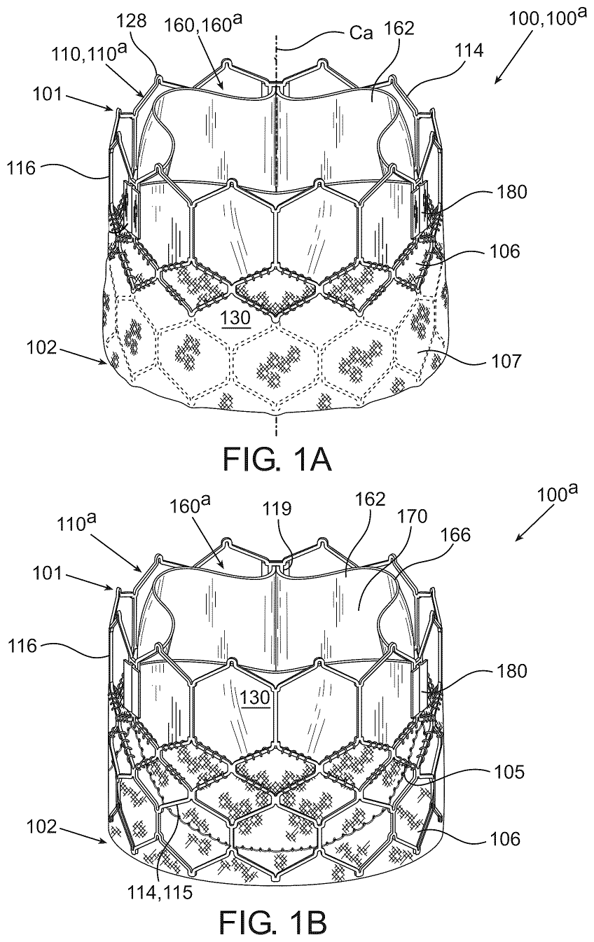

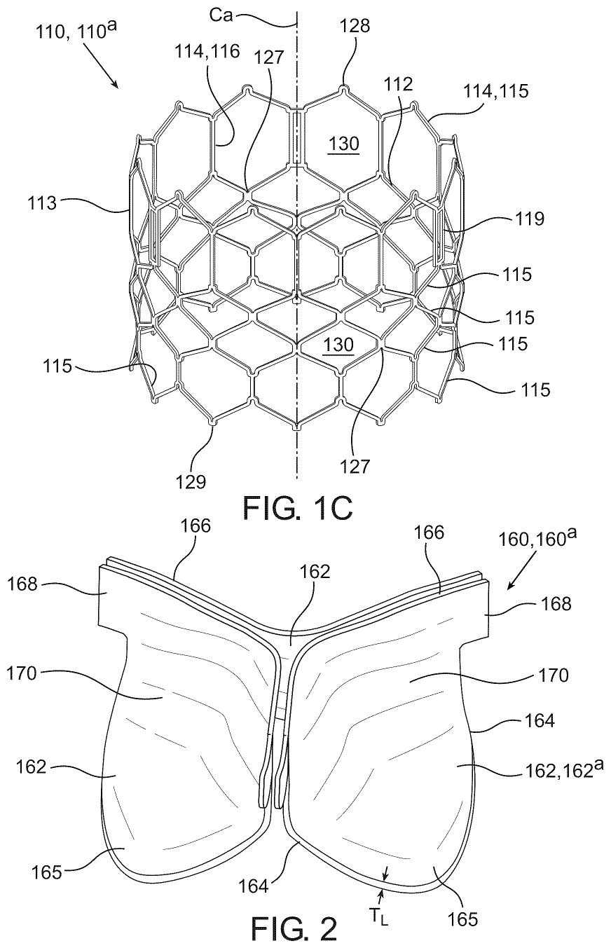

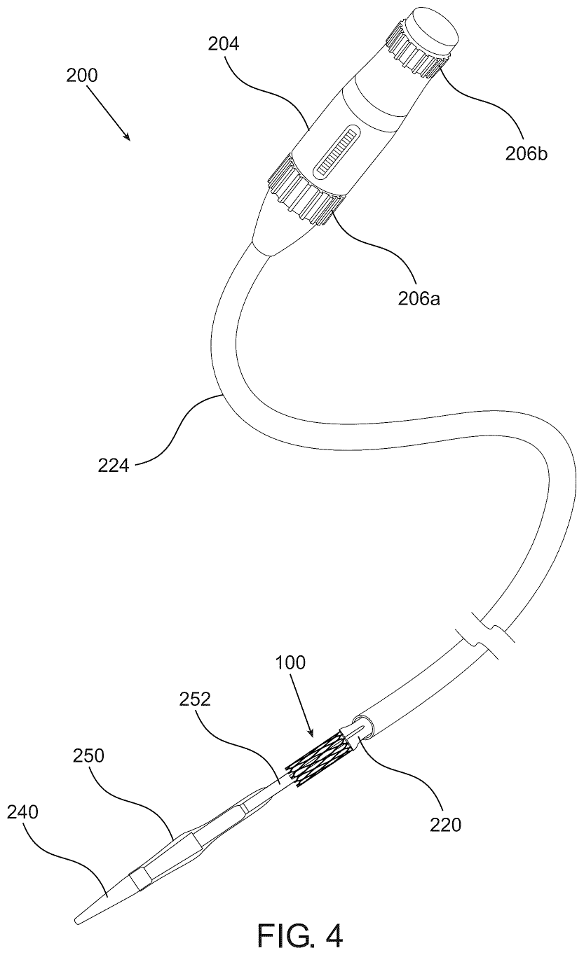

FIGS. 1A and 1B show perspective views of one example of a prosthetic valve 100, with and without an outer skirt 107 surrounding the frame 110, respectively. FIG. 1C shows the frame 110 without any other soft components attached thereto. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valve can be crimped on or retained by an implant delivery apparatus 200 (shown in FIG. 4) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state. A prosthetic valve of the current disclosure (e.g., prosthetic valve 100) may include any prosthetic valve configured to be mounted within the native aortic valve, the native mitral valve, the native pulmonary valve, and the native tricuspid valve.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 1A-1C show an example of a prosthetic valve 100, which can be a balloon expandable valve, illustrated in an expanded state. The prosthetic valve 100 can comprise an outflow end 101 and an inflow end 102. In some instances, the outflow end 101 is the proximal end of the prosthetic valve 100, and the inflow end 102 is the distal end of the prosthetic valve 100. Alternatively, depending for example on the delivery approach of the valve, the outflow end can be the distal end of the prosthetic valve, and the inflow end can be the distal end of the proximal valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The prosthetic valve 100 comprises an annular frame 110 movable between a radially compressed configuration and a radially expanded configuration, and a valvular structure 160 mounted within the frame 110. The frame 110 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 110 can be crimped to a radially compressed state on a balloon catheter, and then expanded inside a patient by an inflatable balloon or equivalent expansion mechanism. Alternatively or additionally, the frame 110 can be made of shape-memory materials such as, but not limited to, nickel titanium alloy (e.g., Nitinol). When constructed of a shape-memory material, the frame 110 can be crimped to a radially compressed state and restrained in the compressed state by insertion into a shaft or equivalent mechanism of a delivery apparatus.

In the example illustrated in FIGS. 1A-1C, the frame 110 is an annular, stent-like structure comprising a plurality of intersecting struts 114. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 114 may be any elongated member or portion of the frame 110. The frame 110 can include a plurality of strut rungs that can collectively define one or more rows of cells 130. The frame 110 can have a cylindrical or substantially cylindrical shape having a constant diameter from the inflow end 102 to the outflow end 101 as shown, or the frame can vary in diameter along the height of the frame, as disclosed in U.S. Pat. No. 9,155,619, which is incorporated herein by reference.

The end portions of the struts 114 are forming apices 128 at the outflow end 101 and apices 129 at the inflow end 102. The struts 114 can intersect at additional junctions 127 formed between the outflow apices 128 and the inflow apices 129. The junctions 127 can be equally or unequally spaced apart from each other, and/or from the apices 128, 129, between the outflow end 101 and the inflow end 102.

The struts 114 can include a plurality of angled struts 115 and vertical or axial struts 116. FIGS. 1A-1C show an exemplary prosthetic valve 100 that can be representative of, but is not limited to, a balloon expandable prosthetic valve. The frame 110 of the prosthetic valve 100 illustrated in FIG. 1C comprises rungs of angled struts 115 and axial struts 116 disposed between some of the rungs of the angled struts. In such implementations of the frame, the struts can be pivotable or bendable relative to each other, so as to permit frame expansion or compression. For example, the frame 110 can be formed from a single piece of material, such as a metal tube, via various processes such as, but not limited to, laser cutting, electroforming, and/or physical vapor deposition, while retaining the ability to collapse/expand radially in the absence of hinges and like.

Each strut 114 can define a strut inner surface 112 facing the valve central longitudinal axis Ca, and an opposite outer surface 113 facing away from the axis Ca. The inner surfaces 112 of the struts 114 collectively define an inner surface of the frame 110. The outer surfaces 113 of the struts 114 collectively define an outer surface of the frame 110. In some implementations, a strut 114 can be formed to have a rectangular cross-section, defining a struts width W_{S} and a strut thickness Ts (shown, for example, in FIG. 10). Strut thickness Ts is measured between the strut inner surface 112 and the strut outer surface 113. Strut width Ws is measured between the opposing surfaces of the strut 114 that extend between its inner 112 and outer 113 surfaces.

A conventional valvular structure 160, shown also for example in FIG. 2, can include a plurality of leaflets 162 (e.g., three leaflets), positioned at least partially within the frame 110, and configured to regulate flow of blood through the prosthetic valve 100 from the inflow end 102 to the outflow end 101. While three leaflets 162 arranged to collapse in a tricuspid arrangement, are shown in the example illustrated in FIGS. 1A-1B and 2, it will be clear that a prosthetic valve 100 can include any other number of leaflets 162. Adjacent leaflets 162 can be arranged together to form commissures 180 that are coupled (directly or indirectly) to respective portions of the frame 110, thereby securing at least a portion of the valvular structure 160 to the frame 110. In some examples, at least some (e.g., three) of the axial struts 116 can define axially extending window frame portions, also termed commissure windows 119, configured to mount respective commissures 180 of the valvular structure 160. The leaflets 162 can be made from, in whole or part, biological material (e.g., pericardium), bio-compatible synthetic materials, or other such materials. Further details regarding transcatheter prosthetic heart valves, including the manner in which the valvular structures 160 can be coupled to the frame 110 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

As shown for example in FIG. 2, three separate leaflets 162 can collectively define the valvular structure 160 in some cases. Each conventional separate leaflet 162 can have a rounded cusp edge 164 opposite a free edge 166, and a pair of generally oppositely-directed tabs 168 separating the cusp edge 164 and the free edge 166. The cusp edge 164 in such cases forms a single scallop. A cusp end portion 165 can be defined for each leaflet 162 as the portion extending from the cusp edge 164, and can be rounded so as to generally track the shape of the cusp edge 164. Each separate leaflet 162 further comprises an inner surface (not annotated), defined as the surface facing the valve central longitudinal axis Ca, and an outer surface (not annotated), opposite thereto so as to face the frame 110. The leaflet 162 can define a leaflet thickness T_{L}, measured between the leaflet's inner and outer surfaces.

When such leaflets 162 are coupled to the frame and to each other, the lower edge of the resulting valvular structure 160 desirably has an undulating, curved scalloped shape. By forming the leaflets with this scalloped geometry, stresses on the leaflets 162 are reduced which, in turn, improves durability of the prosthetic valve. Moreover, by virtue of the scalloped shape, folds and ripples at the belly of each leaflet, which can cause early calcification in those areas, can be eliminated or at least minimized. The scalloped geometry also reduces the amount of tissue material used to form the valvular structure, thereby allowing a smaller, more even crimped profile at the inflow end of the valve.

The leaflets 162 define a non-planar coaptation plane (not annotated) when their free edges 166 co-apt with each other to seal blood flow through the prosthetic valve 100. Leaflets 162 can be secured to one another at their tabs 168 to form commissures 180 of the valvular structure 160, which can be secured, directly or indirectly, to structural elements connected to the frame 110 or integrally formed as portions thereof, such as commissure posts, commissure windows, and the like. When secured to two other leaflets 162 to form valvular structure 160, the cusp edges 164 of the leaflets 162 collectively form the scalloped line 105 of the valvular structure 160. Each leaflet 162 comprises a leaflet body 170, defined between the line of attachment of the leaflet to the frame, for example along scalloped line 105, and the free edge 166. The leaflet body 170 defines the movable portion of the leaflet 162, free to move toward the frame 110 in an open state of the valvular structure 160, and toward central longitudinal axis Ca to co-apt with other leaflets 162 in a closed state of the valvular structure 160. The cusp end portions 165 of leaflets 162 can be defined as the portions along which the leaflet 162 are coupled to the frame 110 along the cusp edges 164, collectively forming scalloped line 105 of the valvular structure 160, such that the leaflet body 170 can be defines as the movable portion of the leaflet 162 extending from the cusp end portions 165 to the free edge 166.

Various exemplary implementations for various components and devices can be referred to, throughout the specification, with superscripts, for ease of explanation of features that refer to such exemplary implementations. It is to be understood, however, that any reference to structural or functional features of any apparatus, device or component, without a superscript, refer to these features being commonly shared by all specific exemplary implementations that can be also indicated by superscripts. In contrast, features emphasized with respect to an exemplary implementation of any assembly, apparatus or component, referred to with a superscript, may be optionally shared by some but not necessarily all other exemplary implementations. For example, prosthetic valve 100^{a} is an exemplary implementation of prosthetic valve 100, and thus includes all of the features described for prosthetic valve 100 throughout the current disclosure, except that while the leaflets 162 of a prosthetic valve 100 can be attached to the frame 110 along their cusp end portions 165 in any manner, the leaflets 162 of prosthetic valve 100^{a} are sutured to an inner skirt, which is in turn attached to the frame of the valve 100^{a}.

In some examples, the prosthetic valve 100 can further comprise at least one skirt or sealing member. FIGS. 1A-1B show an example of a prosthetic valve 100^{a} that includes an inner skirt 106, which can be secured to the inner surface of the frame 110. Such an inner skirt 106 can be configured to function, for example, as a sealing member to prevent or decrease perivalvular leakage. An inner skirt 106 can further function as an anchoring region for valvular structure 160^{a} to the frame 110, and/or function to protect the leaflets 162 against damage which may be caused by contact with the frame 110, for example during valve crimping or during working cycles of the prosthetic valve 100. FIG. 1B shows an inner skirt 106 disposed around and attached to the inner surface of frame 110, wherein the valvular structure 160^{a} is sutured to the inner skirt 106 along scalloped line 105. Additionally, or alternatively, the prosthetic valve 100 can comprise an outer skirt 107 mounted on the outer surface of frame 110, configure to function, for example, as a sealing member retained between the frame 110 and the surrounding tissue of the native annulus against which the prosthetic valve is mounted, thereby reducing risk of paravalvular leakage (PVL) past the prosthetic valve 100.

Any of the inner skirt 106 and/or outer skirt 107 can be made of various suitable biocompatible materials, such as, but not limited to, various synthetic materials (e.g., PET) or natural tissue (e.g., pericardial tissue). In some cases, the inner skirt 106 can be formed of a single sheet of material that extends continuously around the inner surface of frame 110. In some cases, the outer skirt 107 can be formed of a single sheet of material that extends continuously around the outer surface of frame 110.

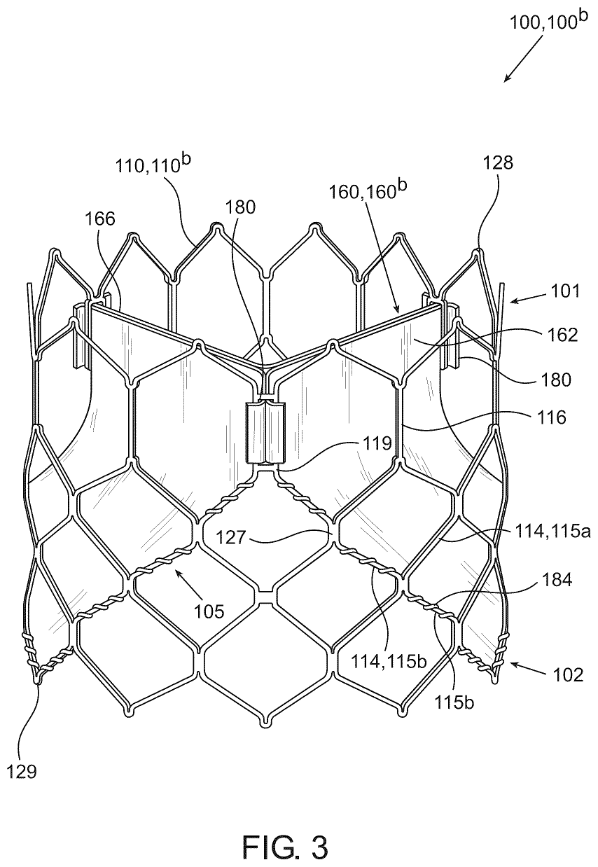

FIG. 3 shows an exemplary prosthetic valve 100^{b}. Prosthetic valve 100^{b} is an exemplary implementation of prosthetic valve 100 and/or 100^{a}, and thus includes all of the features described for prosthetic valve 100 and/or 100^{a} throughout the current disclosure, except that instead of the valvular structure 160^{a} of prosthetic valve 100^{a} anchored to an inner skirt 106, which in turn is coupled to the frame 110, the valvular structure 160^{b} of prosthetic valve 100^{b} is sutured directly to the frame 110^{b}. For example, as shown in FIG. 3, each leaflet 162 can be directly sutured to struts 114 of the frame 110^{b} by one or more sutures 184, instead of via inner skirt 106. While not shown in FIG. 3, the prosthetic valve 100^{b} can further include an outer skirt 107 disposed around the frame 110, similarly to prosthetic valve 100^{a}.

The frame 110^{b} shown in FIG. 3 can comprise a plurality of struts 114, such as angled struts 115, including a group of first struts 115a to which the leaflets 162 are not sutured along their cusp end portions 165, and a group of second struts 115b to which the cusp end portions 165 of the leaflets 162 are sutured, forming a scalloped line 105 along selected angled struts 115b.

FIG. 4 illustrates a delivery apparatus 200, according to an exemplary configuration, adapted to deliver a prosthetic valve 100 according to any example described herein. It should be understood that the delivery apparatus 200 can be used to implant prosthetic devices other than prosthetic valves, such as stents or grafts.

The delivery apparatus 200 includes a handle 204 and a balloon catheter 252 having an inflatable balloon 250 mounted on its distal end. The prosthetic valve 100 can be carried in a crimped state over the balloon catheter 252. Optionally, an outer delivery shaft 224 can concentrically extend over the balloon catheter 252, and a push shaft 220 can be disposed over the balloon catheter 252, optionally between the balloon catheter 252 and the outer delivery shaft 224.

The outer delivery shaft 224, the push shaft 220, and the balloon catheter 252, can be configured to be axially movable relative to each other. For example, a proximally oriented movement of the outer delivery shaft 224 relative to the balloon catheter 252, or a distally oriented movement of the balloon catheter 252 relative to the outer delivery shaft 224, can expose the prosthetic valve 100 from the outer delivery shaft 224. The delivery apparatus 200 can further include a nosecone 240 carried by a nosecone shaft (hidden from view in FIG. 4) extending through a lumen of the balloon catheter 252.

The proximal ends of the balloon catheter 252, the outer delivery shaft 224, the push shaft 220, and optionally the nosecone shaft, can be coupled to the handle 204. During delivery of the prosthetic valve 100, the handle 204 can be maneuvered by an operator (e.g., a clinician or a surgeon) to axially advance or retract components of the delivery apparatus 200, such as the nosecone shaft, the balloon catheter 252, the outer delivery shaft 224, and/or the push shaft 220, through the patient's vasculature, as well as to inflate the balloon 250 mounted on the balloon catheter 252, so as to expand the prosthetic valve 100, and to deflate the balloon 250 and retract the delivery apparatus 200 once the prosthetic valve 100 is mounted in the implantation site.

The handle 204 can include a steering mechanism configured to adjust the curvature of the distal end portion of the delivery apparatus 200. In the illustrated example, the handle 204 includes an adjustment member, such as the illustrated rotatable knob 206a, which in turn is operatively coupled to the proximal end portion of a pull wire. The pull wire can extend distally from the handle 204 through the outer delivery shaft 224 and has a distal end portion affixed to the outer delivery shaft 224 at or near the distal end of the outer delivery shaft 224. Rotating the knob 206a can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of the push shaft 220 relative to the balloon catheter.

The prosthetic valve 100 can be carried by the delivery apparatus 200 during delivery in a crimped state, and expanded by balloon inflation to secure it in a native heart valve annulus. In an exemplary implantation procedure, the prosthetic valve 100 is initially crimped over the balloon catheter 252, proximal to the inflatable balloon 250. Because prosthetic valve 100 is crimped at a location different from the location of balloon 250, prosthetic valve 100 can be crimped to a lower profile than would be possible if it was crimped on top of balloon 250. This lower profile permits the clinician to more easily navigate the delivery apparatus 200 (including crimped prosthetic valve 100) through a patient's vasculature to the treatment location. The lower profile of the crimped prosthetic valve is particularly helpful when navigating through portions of the patient's vasculature which are particularly narrow, such as the iliac artery.

The balloon 250 can be secured to balloon catheter 252 at its balloon proximal end, and to either the balloon catheter 252 or the nosecone 240 at its distal end. The distal end portion of the push shaft 220 is positioned proximal to the outflow end 101 of the prosthetic valve 100.

When reaching the site of implantation, and prior to balloon inflation, the push shaft 220 is advanced distally, allowing its distal end portion to contact and push against the outflow end of prosthetic valve 100, pushing the valve 100 distally therewith. The distal end of push shaft 220 is dimensioned to engage with the outflow end of the prosthetic valve 100 in a crimped configuration of the valve. In some implementations, the distal end portion of the push shaft 220 can be flared radially outward, to terminate at a wider-diameter that can contact the prosthetic valve 100 in its crimped state. Push shaft 220 can then be advanced distally, pushing the prosthetic valve 100 therewith, until the crimped prosthetic valve 100 is disposed around the balloon 250, at which point the balloon 250 can be inflated to radially expand the prosthetic valve 100. Once the prosthetic valve 100 is expanded to its functional diameter within a native annulus, the balloon 250 can be deflated, and the delivery apparatus 200 can be retrieved from the patient's body.

In particular implementations, the delivery apparatus 200 with the prosthetic valve 100 assembled thereon, can be packaged in a sterile package that can be supplied to end users for storage and eventual use. In particular implementations, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 100 and the delivery apparatus 200, can be free of any liquid. Methods for treating tissue leaflets for dry storage are disclosed in U.S. Pat. Nos. 8,007,992 and 8,357,387, both of which documents are incorporated herein by reference.

Conventional prosthetic valves, such as valve 100^{a} or 100^{b}, include a valvular structure 160 with the leaflets 162 that can be either sutured to an inner skirt 106, as illustrated and described for a prosthetic valve 100^{a}, or sutured directly to struts 114 of frame 110, as illustrated and described for a prosthetic valve 100^{b}. The suturing process tends to be labor-intensive, and the quality of the result may depend on the skill-level of the assembler. Moreover, the tension applied to the suture may not be well controlled, which can effect local geometries of the leaflets 162. All of these factors may adversely impact the functionality and structural integrity of the valvular structure 160 in the long term. Disclosed herein are devices and method for securing the leaflets 162, such as along their cusp end portions 165, to the frame 110 of prosthetic valve 100, in a manner that obviates the use of sutures stitched through the leaflet material, which can substantially reduce process times and the difficulty valve assembly procedures. In some examples, the term “cusp end portion” refers to a portion of the leaflet extending to a distance of about twice the strut width W_{S}. In some examples, the term “cusp end portion” refers to a portion of the leaflet extending to a distance of about three times the strut width W_{S}. In some examples, the term “cusp end portion” refers to a portion of the leaflet extending to a distance of about four times the strut width W_{S}.

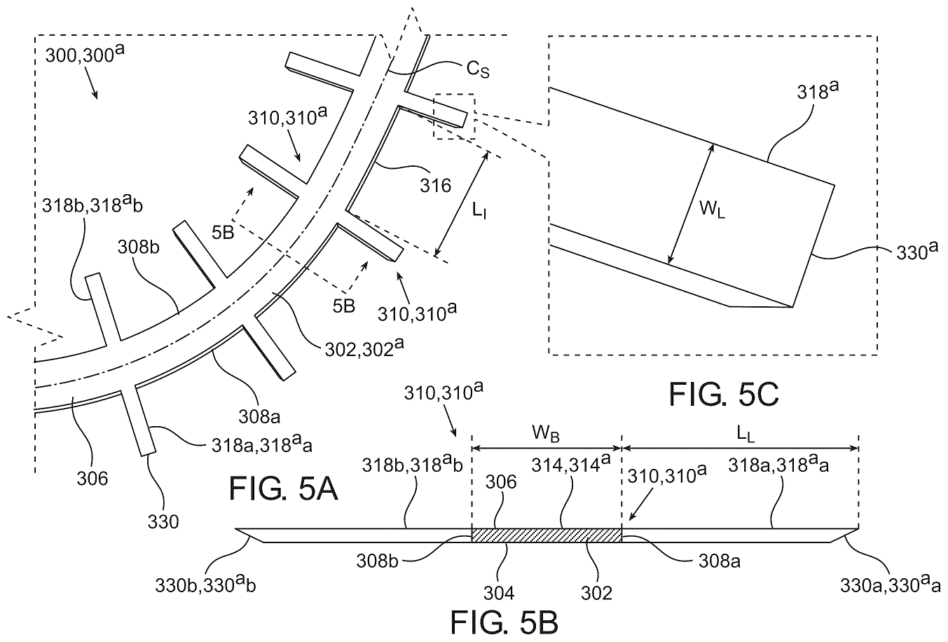

FIG. 5A shows a portion of an exemplary stapling member 300 that can be used for coupling a leaflet 162 to one or more struts 114 of a frame 110, illustrated in a flattened configuration. FIG. 5B is a sectional view taken along line 5B-5B of FIG. 5A. Stapling member 300 comprises a spine 302 extending along stapling member axis Cs, and a plurality of stapling portions 310 separated from each other by intermediate portions 316 of the spine 302. The spine 302 defines a spine first edge 308a, a spine second edge 308b, a spine inner surface 304 extending between the spine first and second edges 308a, 308b, and a spine outer surface 306 opposite to the spine inner surface 304.

Each stapling portion 310 comprises two legs 318 extending from the opposite edges 308 of the spine, such as a first leg 318a extending from the spine first edge 308a and a second leg 318^{b} extending from the spine second edge 308b, and a backspan 314 extending between the first and second legs 318a, 318^{b}. The backspan can be perpendicular to the stapling member axis Ca, such that both legs 318a, 318^{b} extend from the backspan at opposite sides of the stapling member axis Ca. FIG. 5C shows an enlarged view of an end portion of a leg 318. Each leg 318 can have a wide W_{L} defined in a direction which is generally parallel to the direction of stapling member axis Ca. Each leg 318 terminates at a tip 330, which can be a relatively sharp tip configured to penetrate through the thickness of a leaflet 162, such as through a pericardial tissue the leaflet 162 can be formed of. As shown in FIG. 5B, the backspan 314 can define a backspan width W_{B} between both of the spine edges 308a, 308b. Each leg 318 can have a length L_{L} between the corresponding spine edge 308 and the tip 330. In some examples, the backspan width W_{B} is at least as great as the strut width W_{S}.

In some examples, the tip 330 can define a slanted or beveled surface. FIGS. 5A-5C show an exemplary stapling member 300^{a}, which is an exemplary implementation of stapling member 300, and thus includes all of the features described for stapling member 300 throughout the current disclosure, except that the tip 330^{a} defines a slanted surface between the spine inner 304 and outer 306 surface.

Stapling member 300 can be formed, in some examples, as a unitary component, such as be being stamped, laser cut, and the like, from a plate, to form the desired shape of the stapling member 300. In such examples, the spine 302 (including intermediate portions 316 thereof) and the stapling portions 310 are integrally formed. FIG. 5A shows an optional form the stapling member 300 can be provided in after being cut (e.g., laser cut), wherein the legs 318 are co-planar along their entire lengths L_{L} with the spine 302 and/or the intermediate portions 316. While the legs 318 and the spine 302 are shown to have rectangular cross-sectional shapes, it is to be understood that any of the legs 318, the spine 302, the intermediate portions 316, and/or backspans 314, can have any other cross-sectional shape, such as square shape, rhomboid, triangular, circular, elliptical, and the like. In some implementations, cross-sectional shapes of the spine 302 and/or backspans 314 defining a flat spine outer surface 306 can be advantageous in that the flat surface provides increased contact area with the leaflet 162, when pressed against the leaflet to couple it to the frame, as opposed to round surface configurations.

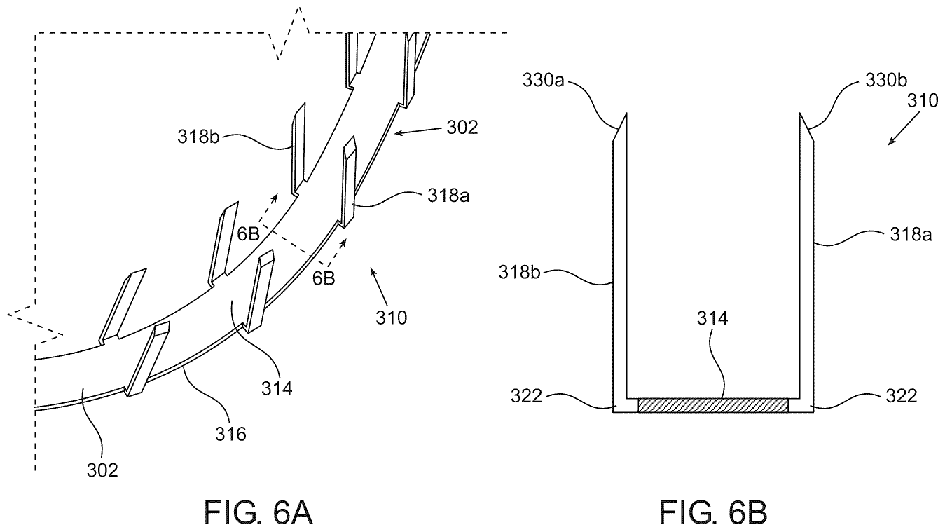

FIG. 6A shows the stapling member 300 in a first bent configuration, wherein the legs 318 are bent at first bends 322 relative to backspans 314. FIG. 6B is a sectional view taken along line 6B-6B of FIG. 6A. As shown, each leg 318 is bent at a first bend 322, relative to the backspan 314, for example at a right angle. The legs 318 are bend outwards, away from the spine inner surface 304, such that each stapling portion 310 can be generally U-shaped. While shown to bend at right angles, such that both legs 318a, 318^{b} are generally parallel to each other between first bends 322 and tips 330, it is to be understood that in some implementations, the legs 318 can be bent at other angles, which can be more or less than 90°.

It is to be understood that any reference throughout the current disclosure to right angles, may refer not only to 90°, but rather to angles approximating this value within a range of ±15°, meaning that any reference to a right angle can refer to an angle in the range of 75°-105°.

Thus, in some implementations, the stapling member 300 can be manufactured from flat metal stock, as shown in FIG. 5A, and then bent to the first bent configuration, as shown in FIG. 6A. The stapling member 300 can be made of plastically-deformable materials such as, but not limited to, stainless steel or a nickel based alloy (e.g., a cobalt-chromium or a nickel-cobalt-chromium alloy such as MP35N alloy), titanium, and the like.

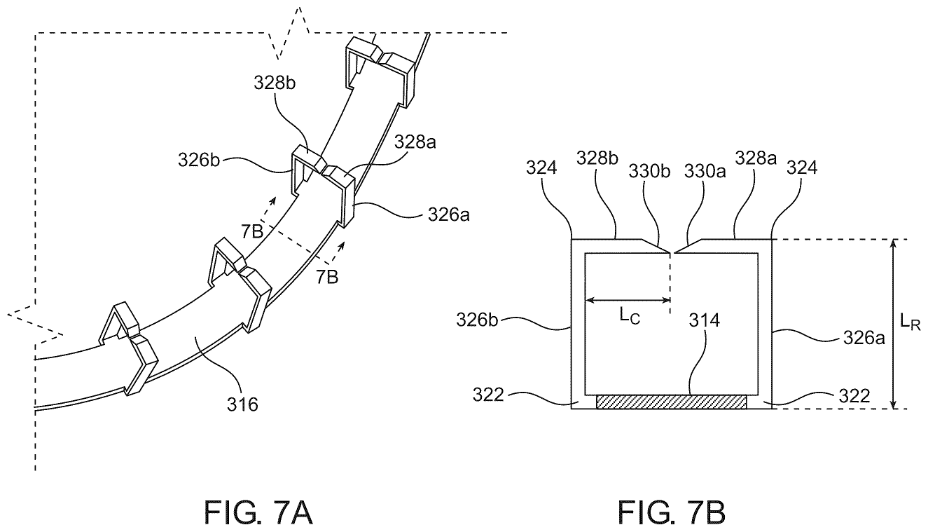

FIG. 7A shows the stapling member 300 in a second bent configuration, wherein the legs 318 are further bent towards each other at second bends 324. FIG. 7B is a sectional view taken along line 7B-7B of FIG. 6A. As shown, each leg is bent at a second bend 324, between the first bend 322 and the tip 330, dividing the leg 318 into a radial portion 326 extending between the first bend 322 and the second bend 324, and a clinch portion 328 extending between the second bend 324 and the tip 330. The radial portion 326 can extend along a radial portion length L_{R}, and the clinch portion 328 can extend along a clinch portion length L_{C}, such that L_{R}+L_{C}=L_{L}.

The first and second clinch portions 328a, 328^{b} extend towards each other, and can be generally parallel to the backspan 314. In some examples, as shown in FIGS. 5A-7B, the legs 318 of each stapling portion 310 are aligned with each other, such that their tips 330 are positioned in front of each other in the second bent configuration. In such cases, since the clinch portions 328 are coaxial, it may be desirable to design the legs 318 such that the combined sum of lengths L_{C} of both clinch portions 328 is equal to or less than the backspan width W_{B} and/or less to prevent them from engaging each other when bent to the second bent configuration. In some examples, the combined sum of lengths L_{C} of both clinch portions 328 is equal to or less than the strut width W_{S}.

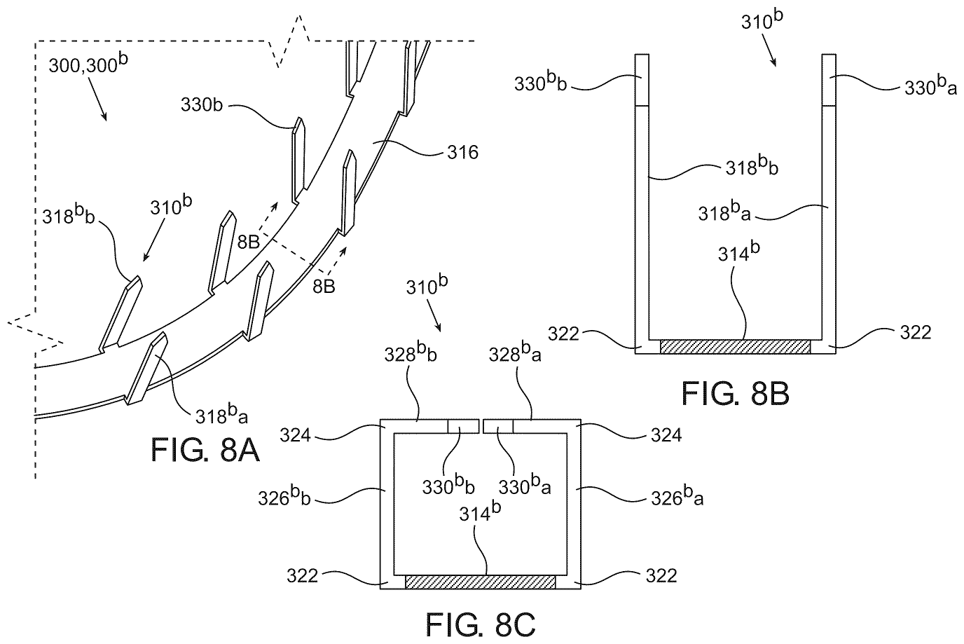

In some examples, the tip 330 can define an arrowhead shape. FIG. 8A shows a portion of an exemplary stapling member 300^{b} in a first bent configuration. FIG. 8B is a sectional view taken along line 8B-8B of FIG. 8A, showing a stapling portion 310^{b} in the first bent configuration. FIG. 8C shows the stapling portion 310^{b} of FIG. 8B in a second bent configuration. Stapling member 300^{b} is an exemplary implementation of stapling member 300, and thus includes all of the features described for stapling member 300 throughout the current disclosure, except that the tip 330^{b} of each leg 318^{b} defines an arrowhead shape.

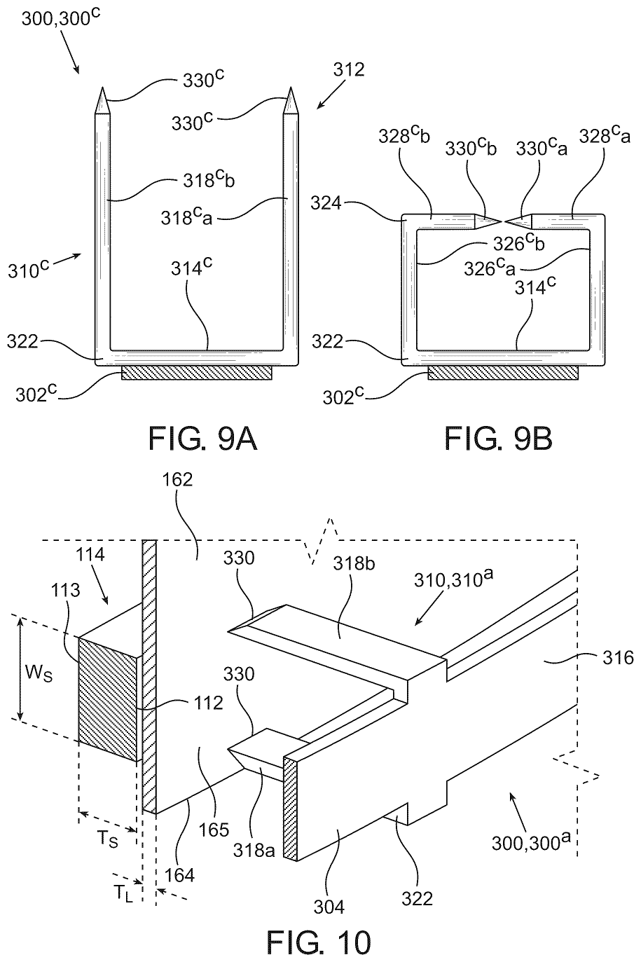

In some examples, a stapling member 300 can include stapling elements 312 coupled to the spine 302. FIG. 9A is a sectional view showing a stapling portion 310^{c} of a stapling member 300^{c} in a first bent configuration. FIG. 9B shows the stapling portion 310^{c} of FIG. 9A in a second bent configuration. As shown, stapling portion 310^{e} comprises a stapling element 312 that includes the backspan 314^{c} and legs 318^{c}, wherein the stapling portion 310^{c} is attached, at its backspan 314^{c}, to the spine 302^{c}. The spine 302^{c} and stapling elements 312 can be provided as separate components, which can be attached to each other by gluing, welding, and the like. The spine 302^{c} and stapling elements 312 can be formed of different or similar materials. Providing separately formed stapling elements 312 and spine 302 attachable to each other allows each to be formed with a different shape and cross-section. For example, while spine 302^{c} can be manufactured (e.g., stamped, laser cut, and the like) from a plate (e.g., metal plate), having a rectangular cross section as shown in FIGS. 9A-9B, each stapling element 312 can be formed from a wire (e.g., metal wire) having a circular cross-section. In some examples, the tip 330 can conical, as shown for tips 330^{c} in FIGS. 9A-9B.

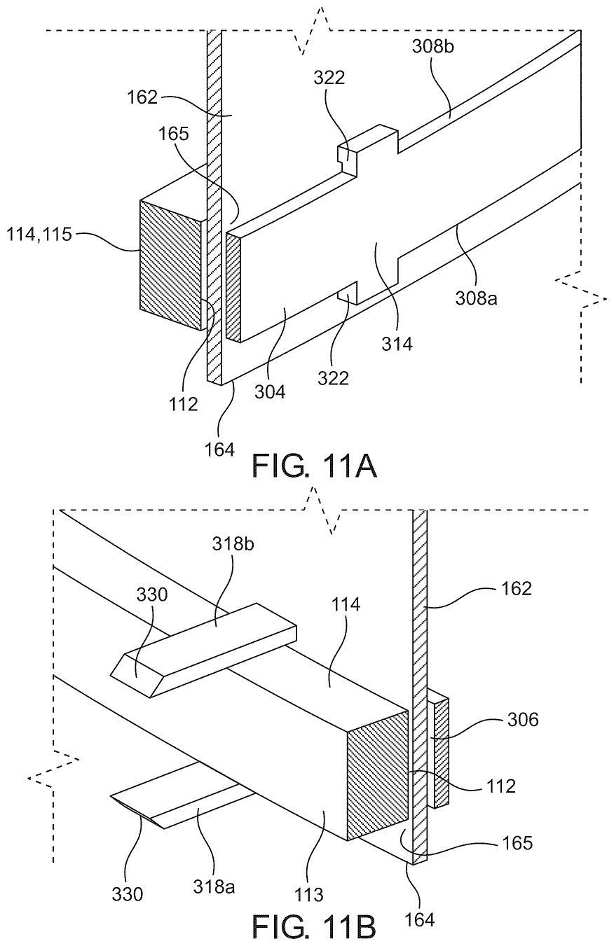

FIGS. 10-12 show steps in a method of coupling a leaflet 162 to strut 114 by stapling member 300. As shown in FIG. 10, a leaflet's cusp end portion 165 can be placed adjacent to one or more struts 114 of the frame 110 (a portion of one leaflet 162 next to a portion of one strut 114 is shown in FIG. 10), such that the cusp edge 164 can be somewhat lower than the strut 114. The leaflet's cusp end portion 165 is placed against the inner surface of the frame 110, such that the leaflet 162 is facing the strut inner surface 112. Stapling member 300, in a first bent configuration thereof, can be then approximated to the leaflet 162 from the inner side, such that the spine inner surface 304 faces the valve central longitudinal axis Ca and the spine outer surface 306 faces the leaflet 162, orienting the legs 318 toward the leaflet 162. As shown in FIGS. 11A-11B, illustrated as viewed from the inner and outer sides of the valve frame, the legs 318 are then pressed into the leaflet 162, causing the tips 330 to pierce through the tissue material, and the legs 318 penetrate into leaflet 162 and extend outward beyond the strut outer surface 113, sandwiching the leaflet 162 between the backspan 314 and the strut 114.

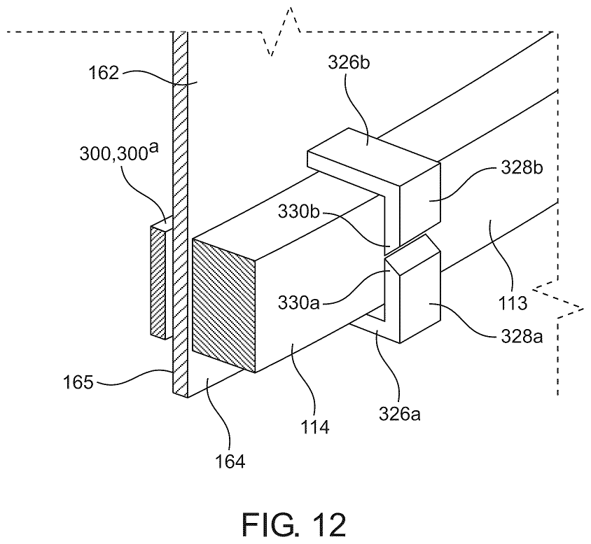

FIG. 12 shows the next step of bending the legs 318 over the outer surface 113 of the strut 114, transitioning the stapling member 300 to the second bent configuration, thus clamping the stapling member 300 over strut 114 to couple the leaflet 162 to the frame 110, for example through the cusp end portions 165. While a stapling member 300^{a} is illustrated in FIGS. 10-12, it is to be understood that this is shown by way of illustration and not limitation, and that a stapling member 300 that includes any other shape of tips 330, such as stapling members 300^{b} or 300^{c} described above, can be similarly used.

In some implementations, as shown for example in FIG. 5A, the stapling member 300 can be generally curved, meaning that it extends along a curved stapling member axis Cs, optionally following a curvature of the cusp edge 164 and/or cusp end portion 165 of leaflet 162. In some implementation, the curved shape of one or more stapling member 300 can follow a scalloped shape of the scalloped line 105, to allow coupling of the valvular structure 160, by one or more stapling members 300, along a generally scalloped-shaped line 105. Intermediate portion 316 can extend along an intermediate portion length L_{I} between adjacent stapling portions 310. Intermediate portion length L_{I} can be measured along the spine first edge 308a, the spine second edge 308b, or the stapling member axis Cs centrally disposed between the spine edges 308a and 308b. Different intermediate portions 316 of the spine 302 can have similar or different lengths L_{I}. For example, intermediate portions 316 extending between subsequent stapling portion 310 configured to couple a leaflet 162 to a section of a strut 114 extending between two neighboring junctions 127 can be shorter in length than intermediate portions 316 that extend along junctions 127 of the frame.

As shown in FIG. 12, the radial portions 326 of legs 318 extend through the thickness of the leaflet 162 and across the strut 114. In some examples, the radial portion length L_{R} is substantially equal to the combined thicknesses of the leaflet 162 and the strut 114 (i.e., L_{R}≈T_{L}+T_{S}). In some examples, the radial portion length L_{R} is within the range of ±20% of the combined thicknesses of the leaflet 162 and the strut 114, such that 0.8 (T_{L}+T_{S})<L_{R}<1.2 (T_{L}+T_{S}).

As shown, the directions of attachment, extending the legs 318 from the inner side of the frame 110 outwards, may be advantageous, resulting in the relatively sharp tips 330 disposed on the outer side of the valve 100, which reduces the risk of these tips 330 being otherwise engaged by movable portions of the leaflets 162 inside the valve 100.

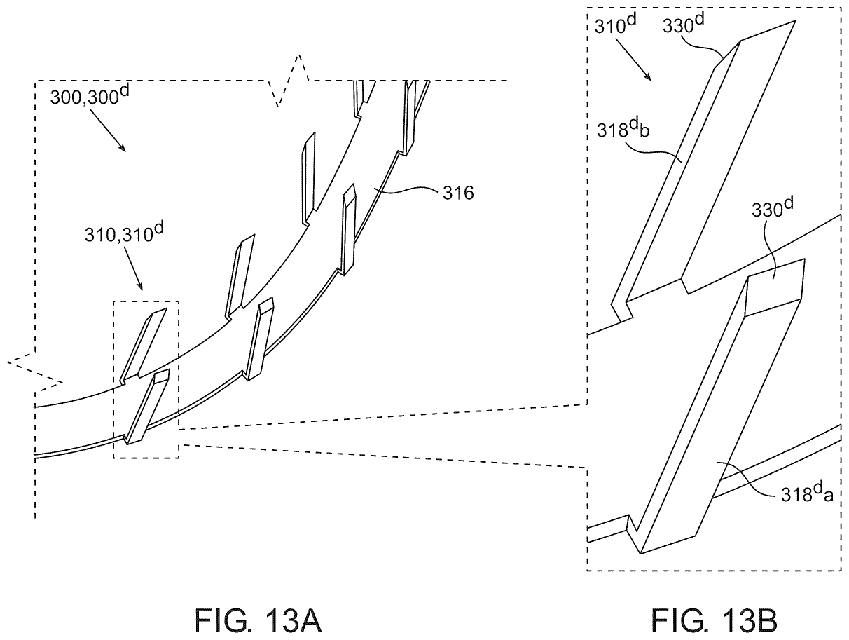

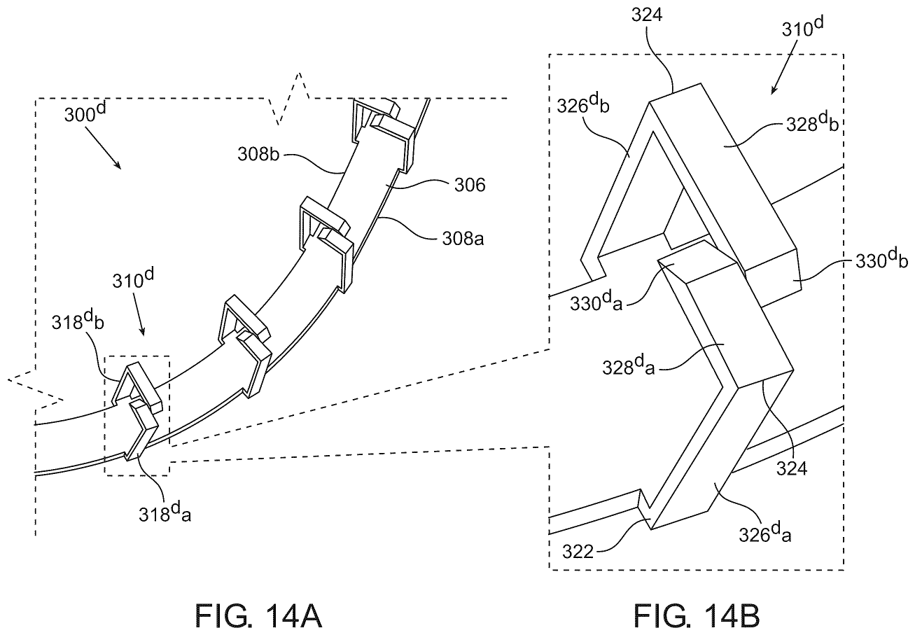

In some examples, the legs 318 of each stapling portion 310 can be offset from each other. FIG. 13A shows a portion of a stapling member 300^{d} in a first bent configuration. FIG. 13B shows an enlarged view of one of the stapling portions 310^{d} of FIG. 13A. FIG. 14A shows the stapling member 300^{d} in a second bent configuration. FIG. 14B shows an enlarged view of one of the stapling portions 310^{d} of FIG. 14A. Stapling member 300^{d} is an exemplary implementation of stapling member 300, and thus includes all of the features described for stapling member 300 throughout the current disclosure, except that the legs 318^{d} of each stapling portion 310^{d} are not aligned with each other, as shown in FIGS. 5A-12, but are rather offset from each other, such that when the legs 318^{d} are bent at second bends 324, the clinch portion 328^{d} do not intersect with each other but rather extend parallel to each other.

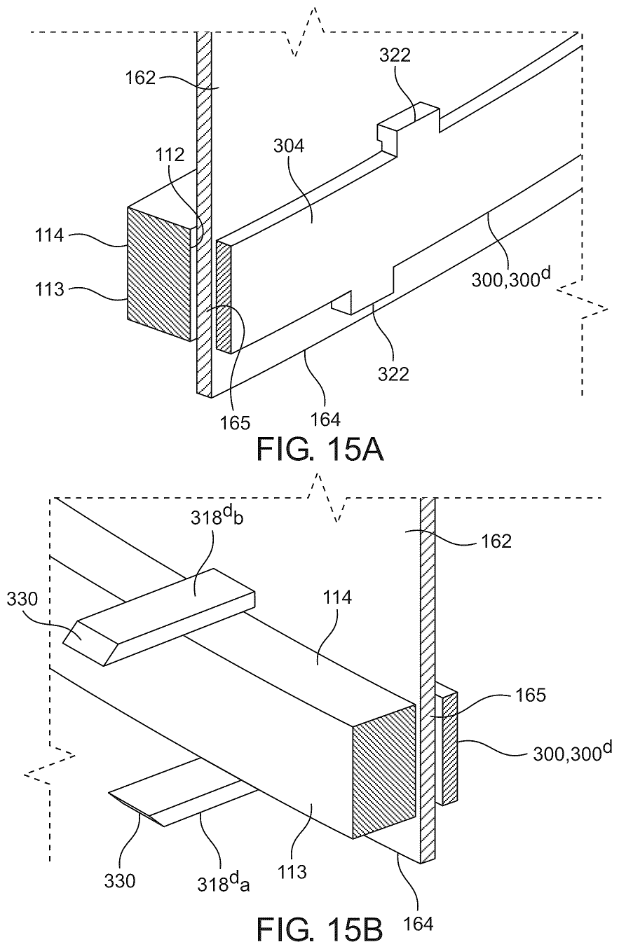

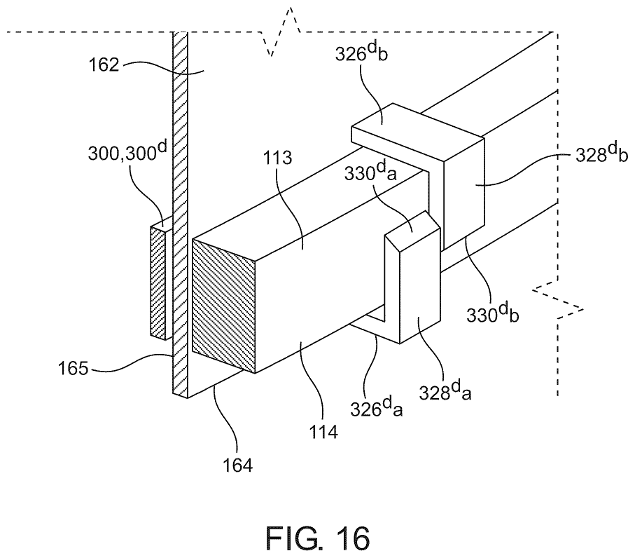

FIGS. 15A-16 show steps in a method of coupling a leaflet 162 to strut 114 by stapling member 300^{d}. As shown in FIGS. 15A-15B, illustrated as viewed from the inner and outer sides of the valve frame, the tips 330^{d} can pierce through the leaflet, such that the legs 318^{d} extend outward through the leaflet 162 and beyond the strut outer surface 113, sandwiching the leaflet 162 between the backspan 314^{d} and the strut 114. As further shown in FIG. 16, the offset legs 318^{d} are then bent over the outer surface 113 of the strut 114, transitioning the stapling member 300^{a} to the second bent configuration, thus clamping the stapling member 300^{d} over strut 114 to couple the leaflet 162 to the frame 110. While a stapling member 300^{d} is illustrated in FIGS. 13A-16 to include tips 330^{d} which are illustrated to include slanted surface, similar to tips 330^{a}, it is to be understood that any other shape of tips can be used with offset legs 3184, including tips forming an arrowhead shape, as illustrated for tip 330^{b}, or conical tips 330^{c}. Similarly, the offset legs 318^{d} can be similarly implemented as offset legs of stapling elements 312 connected to a spine, as described above for stapling members 300^{c}.

Unlike legs 318 which are aligned with each other, limiting the clinch portion length L_{C} as described above, the offset configuration of legs 318^{d} allows the clinch portion 328^{d} to be longer, such that the clinch portion length L_{C} can be, for example, up to the value of the backspan width W_{B}, without the risk of the tips 330^{d} contacting or otherwise interfering with each other. This, in turn, can increase surface contact area between the clinch portions 328^{d} and strut 114, to improve clamping force.

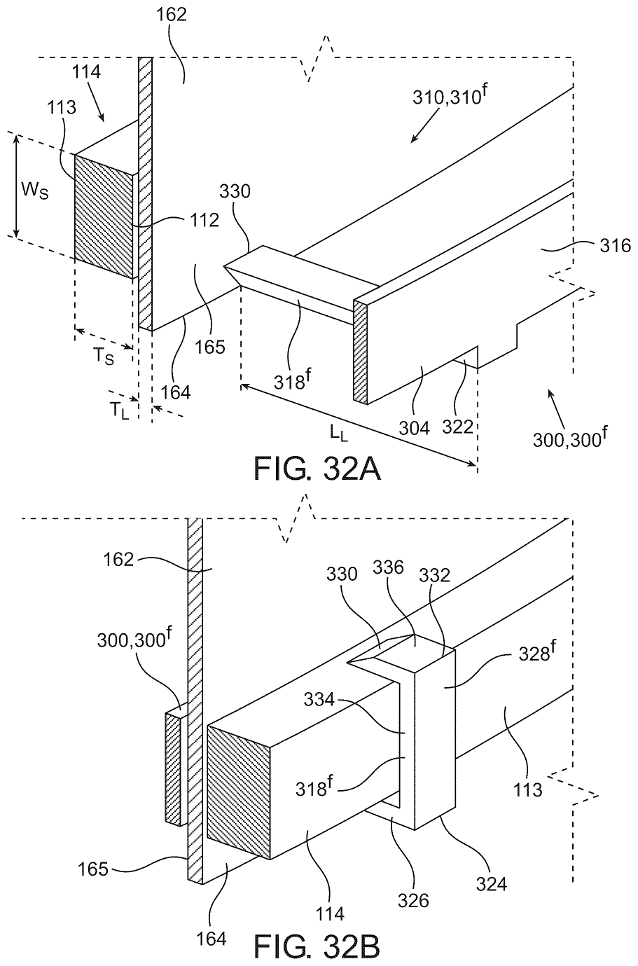

While described to be equipped with tips 330 shaped to penetrate through the thickness of a leaflet 162, in some examples, a coupling member 300 can be equipped with blunt tips. FIG. 31 shows a cusp portion of an exemplary portion of a leaflet 1624, positioned between a strut 114 and exemplary stapling member 300^{e}. Stapling member 300^{e} is an exemplary implementation of stapling member 300, and thus includes all of the features described for stapling member 300 throughout the current disclosure, except that the tips 330^{e} are blunt. The leaflet 162^{e} includes all of the features described for any leaflet 162 throughout the current disclosure, except that the leaflet 162^{d} further comprises pre-formed perforations sized to allow passage of the arms 318 therethrough, such that arms 318^{e} equipped with blunt tips 330^{e} can extend through the leaflet 162^{a} without the need to perforate or cut through the leaflet material. It is to be understood that blunt tips, such as shown for tips 330^{e}, can be implemented for any other example of stapling member 300 disclosed herein.

While stapling portions 310 of various exemplary stapling members 300 are described and shown to include two legs 318, in some examples, a stapling portion 310 can be equipped with a single leg 318. FIG. 32A shows an exemplary stapling member 300^{f} positioned next to a leaflet 162 prior to coupling the leaflet to the strut 114. Stapling member 300^{f} is an exemplary implementation of stapling member 300, and thus includes all of the features described for stapling member 300 throughout the current disclosure, except that the stapling portion 310^{f} includes a single leg 318^{f} instead of two opposing legs. The stapling portion 310^{e} is shown in a first bent configuration in FIG. 32A, with the leg 318^{f} bent at a first bend 322, forming an L-shaped stapling portion 310^{f} in this first bent configuration.

FIG. 32B shows the stapling member 300^{f} of FIG. 32A coupling the leaflet 162 to the strut 114, with the leg 318^{f} wrapped around the strut 114 in the second bent configuration. In some examples, the leg length L_{L} can be greater than the sum of the strut thickness T_{S} and the struts width W_{S} (i.e., L_{L}>T_{S}+W_{S}), such that the leg 318^{f}, in the second bent configuration, can be wrapped around three surfaces of the strut 114, forming not only a second bend 324 but a third bend 332 as well. As shown in FIG. 32B, leg 318^{f} extends radially inwards, along a radial portion 326 thereof, from the first bend 322 (shown in FIG. 32A), through the leaflet 162, toward the strut inner surface 112. The leg 318^{f} is then bent at a second bend 324 to form an axial clinch section 334 extending axially along the strut inner surface 112, and is finally bent at a third bend 332 to extend radially outward (i.e., toward leaflet 162 or strut outer surface 113), forming a radial clinch section 336 extending from the third bend 332 and terminating at the tip 330.

As shown, the tip 300 is facing the leaflet 162 but does not necessarily contact it or re-penetrate it. This means, in such examples, that the leg length L_{L} can be less than the sum of strut width W_{S} and twice the strut thickness T_{S} (i.e., L_{L}<2 T_{S}+W_{S}), resulting in a radial clinch section 336 which is parallel to, but shorter than, the radial portion 326. While a second bent configuration forming a third bend 332 that divides the clinch portion 328 into a clinch axial section 334 and a clinch radial section 336 is shown and described with respect to a stapling portion 310^{f} having a single leg 318^{f}, it is to be understood that other stapling portions 310 described herein to include two legs 310, such as stapling portion 310^{d} of stapling members 300^{d} that include pairs of offset legs 3184, can have leg lengths L_{L} and be bent in a similar manner to that described above with respect to leg 318^{f}. In such cases, the offset nature of a pair of legs allows each to be formed with a length that allows it to be wrapped around three surfaces of the strut, without the legs 318 contacting or otherwise engaging each other. In some examples, a stapling member 300 can be equipped with pairs of legs 318 which are aligned with each other, yet having leg lengths similar to those described above for leg 318^{f}, in which case, some portions of clinch portions 328 of both legs, including the radial clinch sections 336 and/or axial clinch sections 334, may be configured to at least partially overlap over each other in the second bent configuration.

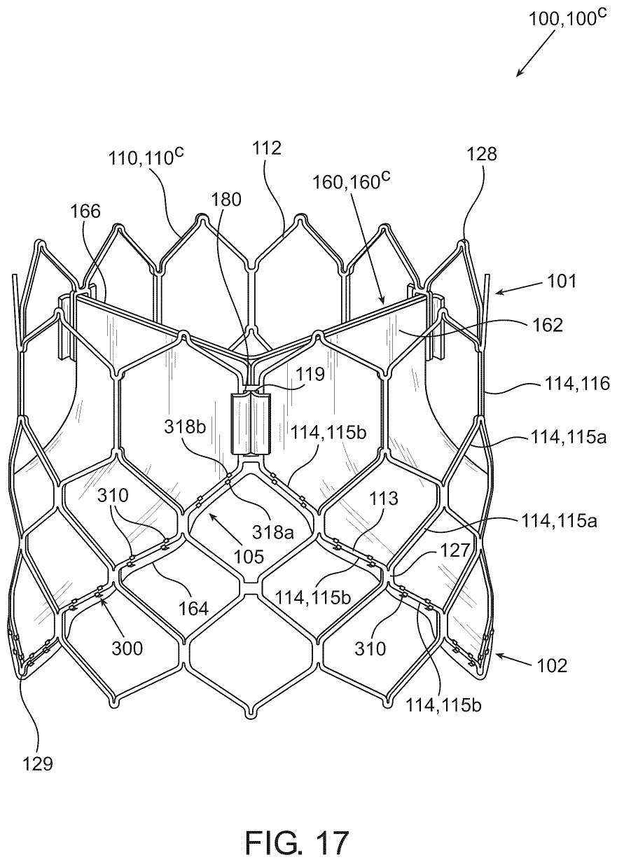

FIG. 17 shows an exemplary prosthetic valve 100^{c}. Prosthetic valve 100^{c} is an exemplary implementation of prosthetic valve 100, and thus includes all of the features described for prosthetic valve 100 throughout the current disclosure, except that the leaflets 162 are coupled to the frame 110^{c}, for example along their cusp end portions 165, via stapling members 300. The frame 110^{c} shown in FIG. 17 can be generally similar to the frame 110^{b}, comprising a plurality of struts 114, such as angled struts 115 that include a group of first struts 115a to which the leaflets 162 are not coupled along their cusp end portions 165, and a group of second struts 115b to which the cusp end portions 165 are coupled, forming a scalloped line 105 along selected angled struts 115b.

Any number of stapling members 300 can be utilized. In some examples, a single stapling member 300 can be utilized to couple all leaflets 162 to the frame 110. For example, a single stapling member 300 can have an undulating or scalloped-shaped extending across the complete circumference of the frame 110. In some examples, the number of stapling members 300 can be similar to the number of the leaflets 162, For example, for a tri-leaflet valvular structure 160^{c}, three cusp-shaped stapling members 300 can be used, each formed to generally follow the shape of the cusp of one of the leaflets 162. In some examples, the number of the stapling members 300 can be twice the number of leaflets 162. For example, for a tri-leaflet valvular structure 160^{c}, six cusp-shaped stapling members 300 can be used, each shaped to extend between an inflow apex 129 and a commissure 180, such that each stapling member 300 is used to couple about half of the cusp end portion 165. In some examples, several stapling members 300 can be used to couple different portions of the cusp edge 164 to the frame 110. For example, separate stapling members 300 can be used to extend over each strut 115a separately.

The shape of the stapling members 300 can generally follow the shape of the struts 115 to which they are coupled. For example, the second struts 115b illustrated in FIG. 17 are shown to extend in a zig-zagged pattern between each inflow apex 129 and a commissure 180, due to the junctions 127 disposed between adjacent struts 115b, causing each second strut 115b to be offset relative to the neighboring second strut 115b. A stapling member 300 configured to extend along more than one strut 115b, can be shaped to exhibit a similar zig-zagged pattern.

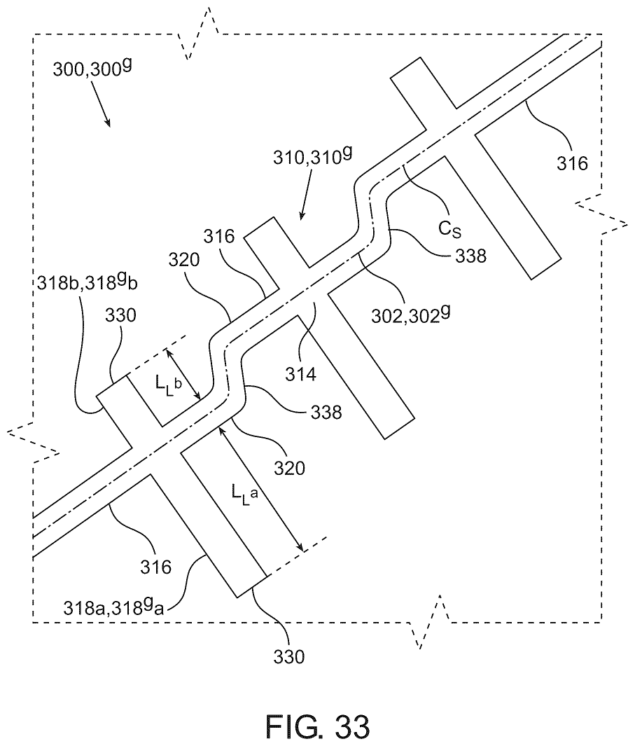

FIG. 33 shows a portion of an exemplary stapling member 3008. Stapling member 300^{g} is an exemplary implementation of stapling member 300, and thus includes all of the features described for stapling member 300 throughout the current disclosure, except that the spine 3028 extends in a zig-zagged pattern, shaped to generally match the zig-zagged pattern of the second struts 115b shown in FIG. 17 for example. In some examples, intermediate portion 316 of spine 3028 includes an offsetting portion 338 disposed between sub-portions 320, wherein the sub-portions 320 can be parallel to each other, yet offset from each other in a direction which is angled relative to the orientation of the sub-portions 320. That is to say, the stapling member axis Cs extends in a zig-zagged pattern, such that the offsetting portions 338 are angled relative to the sub-portions 320. When used to coupled leaflets 162 to the frame 102, the offsetting portions 338 are aligned with corresponding junctions 127, and are shaped and sized to align the sub-portions 320 with the angled struts 115 extending from the junctions 127.

In some examples, the legs 318 of stapling portion 310 can have different leg lengths L_{L}. Stapling portion 3108 is illustrated in FIG. 33 to include a first leg 3188^{g} having a leg length LLa and a second leg 3188^{g} having a leg length L_{L}b, wherein the length of the first leg 318^{g}a is greater than the length of the second leg 3188^{g} (i.e., L_{L}a>L_{L}b). While the legs 318^{g}a and 318^{g}b are illustrated to be aligned with each other in FIG. 33, it is to be understood that this is shown by illustration and not limitation, and that in some examples, legs 318 having unequal leg lengths can be offset from each other, such as shown and described for stapling members 300^{d} for example.

While pairs of legs having unequal lengths is shown in combination with a zig-zagged spine in FIG. 33, it is to be understood that these features can be used in isolation, and combined with features of any other exemplary stapling member disclosed herein. For example, a zig-zagged spine, such as spine 3028, can be used in combination with stapling portions 310 equipped with legs 318 having equal lengths (see FIGS. 5A-16), which can be aligned (see FIGS. 5A-12) or offset legs (see FIGS. 13-16), or stapling members 310 equipped with a single leg 318 (see FIGS. 32A-32B). Similarly, legs having unequal lengths, such as legs 3188, can be implemented with spines which are not necessarily zig-zagged, such as curved spine 302^{a} for example.

Referring back to FIG. 17, While prosthetic valve 100^{c} is shown without an inner skirt 106 or an outer skirt 107, it is to be understood that any of an inner skirt 106 and/or an outer skirt 107 can be added, in which case, the length L_{R} of the radial portions 326 of the legs 318 can be adapted to extend through the thicknesses of the inner skirt 106 and/or outer skirt 107 as well. When an outer skirt 107 is added, the legs 318 can either penetrate through the outer skirt 107 or not. In some examples, the legs 318 extend through the thickness of an outer skirt 107 extending around the frame 110, such that the clinch portions 328 are bent over an outer surface of the outer skirt 107. In some examples, the legs 318 are bent over the struts 115, such that the clinch portions 328 extend over strut outer surfaces 113, and the outer skirt 107 is then attached to the frame 110 around and over the clinch portions 328. Such examples can advantageously conceal the tips 330 from contacting the surrounding anatomy around an implanted prosthetic valve 100.

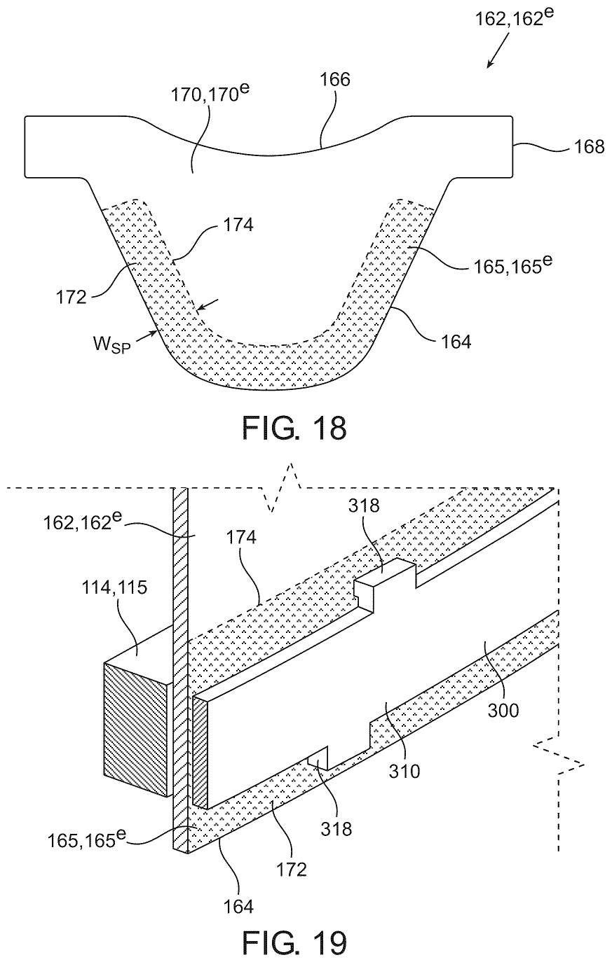

In some examples, the leaflets include increased-stiffness portions through which stapling members can extend to couple the valvular structure to the frame. FIG. 18 shows an example of a leaflet 162^{e}. Leaflet 162^{e} is an exemplary implementation of leaflet 162, and thus includes all of the features described for leaflet 162 throughout the current disclosure, except that leaflet 162^{e} further comprises a stiff portion 172 with a stiffness that is greater than the stiffness of the leaflet body 170. The stiff portion 172 can extend from the cusp edge 164 and terminate at a stiff portion proximal end 174, which can generally track the shape of the cusp edge 164. The stiff portion proximal end 174 can be the border between the leaflet body 170, which is the movable portion of leaflet 162^{e}, and the stiff portion 172, defining stiff portion width W_{SP} between the cusp edge 164 and the stiff portion proximal end 174. The stiff portion 172 can serve as the cusp end portion 165^{e} of leaflet 162^{e}.

The stiff portion 172 can extend all the way up to the tabs 168, or it can terminate below the level of the tabs, as illustrated in FIG. 18. In some examples, the stiff portion proximal end 174 is parallel to the cusp edge 164, such that the stiff portion width W_{SP} is uniform along the stiff portion 172. In other examples, the stiff portion width W_{SP} can vary, for example from a wider width at the lower tip of the leaflet to a narrower width at the upper ends of the stiff portion 172, closer to tabs 168.

Leaflet 162^{e} can be formed, in some examples, from a unitary continuous piece of material, wherein different portions of the leaflet 162^{e} can be treated to have different material properties. Leaflet 162^{e} can be formed from natural tissue, such as bovine pericardium, undergoing biological treatment procedures that can include subjecting the tissue to cross-linking agents, which can influence its final material properties. In such an example, each of stiff portion 172 can be subjected to a different biological procedure than the remainder of the leaflet, including leaflet body 170 and optionally tabs 168, such as: being subjected to different cross-linking agents, subjected to such agents for different time durations, or other procedural variations, adapted to result in stiff portion 172 that can be stiffer than the leaflet body 170. Thus, the leaflet body 170 of leaflet 162^{e}, which has material properties comparable to those of the leaflet body 170 of conventional leaflets 162 (including, for example, leaflet 162^{a}), is the portion of the leaflet 162^{e} which is not directly attached to the frame 110, but is rather configured to freely move toward the frame 110 in an open state of the valvular structure 160^{e}, and toward valve central longitudinal axis Ca in a closed state of the valvular structure 160^{e}.

As mentioned, leaflet 162^{e} can optionally exhibit a different degree of cross-linking in different portions thereof, such that the stiff portion 172 can be more highly cross-linked than the leaflet body 170. This can be achieved, in some implementations, by cross-linking the stiff portion 172 with a first cross-linking agent or solution and cross-linking the leaflet body 170 with a second cross-linking agent. Alternatively or additionally, the stiff portion 172 can be cross-linked during a longer period of time relative to the leaflet body 170. Cross-linking agents can include, but are not limited to, divinyl sulfone (DVS), polyethylene glycol divinyl sulfone (VS-PEG-VS), hydroxyethyl methacrylate divinyl sulfone (HEMA-DIS-HEMA), formaldehyde, glutaraldehyde, aldehydes, isocyanates, alkyl and aryl halides, imidoesters, N-substituted maleimides, acylating compounds, carbodiimide, hexamethylene diisocyanate, 1-Ethyl-3-[3-dimethylaminopropyl] carbodiimide hydrochloride (EDC or EDAC), hydroxychloride, N-hydroxysuccinimide, and combinations thereof.

The leaflet body 170 can be designed to have material properties substantially similar to those of conventional leaflets 162 described above with respect to FIGS. 1A-2, allowing this portion of the leaflet to move between the closed and open states in a similar manner. The stiff portion 172 can be stiffer compared to the leaflet body 170, facilitating easier penetration of stapling members 300 utilized to couple these portions to the frame during valve assembly, and improving engagement retention with the stapling members 300. The stiff portion 172 can be semi-rigid, meaning that it is stiffer or more rigid than the leaflet body 170, yet flexible enough to transition between the crimped and expanded configurations of the prosthetic valve without experiencing material failure and without resisting such transitions of the valve.

In some examples, the ultimate tensile stress of the stiff portion 172 is at least 1.5 times greater than the ultimate tensile stress of the leaflet body 170. In some examples, the ultimate tensile stress of the stiff portion 172 is at least 2 times greater than the ultimate tensile stress of the leaflet body 170. For example, for a leaflet body 170 having an ultimate tensile stress of about 1 MPa (Megapascal), stiff portion 172 can have an ultimate tensile stress which is greater than 1.5 MPa, and in some cases, greater than 2 MPa.

In some examples, the stiff portion 172 obtains a load at failure which is at least 2 times greater than the load at failure obtained by the leaflet body 170. In some examples, the stiff portion 172 obtains a load at failure which is at least 3 times greater than the load at failure obtained by the leaflet body 170. For example, for a leaflet body 170 that obtains a load at failure of about 3 N (Newton), the stiff portion 172 can obtain a load at failure which is greater than 6 N, and in some cases, greater than 9 N.

The stiffness of the stiff portion 172 can be homogenous or non-homogenous. For example, the stiffness of the stiff portion 172 can be homogenous between the cusp edge 164 and the stiff portion proximal end 174, or it can vary from higher stiffness at the cusp edge to lower stiffness at the stiff portion proximal end 174. In such cases, the minimal stiffness of the stiff portion 172 will be equal to or greater than the stiffness of the leaflet body 170.

In some examples, the entire leaflet 162^{e} can be provided as a unitary continuous material, wherein the stiff portion 172 can be reinforced by coating with a reinforcing layer, or otherwise attached along a surface thereof to a reinforcing layer that imparts additional stiffness thereto, such as a polymeric layer or any other suitable layer. The stiffness of any portion of a valvular structure can be measured by following the general procedures set forth in ASTM D790.

In some examples, the leaflet 162^{e} can be formed from two different materials, joined to each other along stiff portion proximal end 174, which serves in such cases as an attachment line 174 (achieved, for example, by suturing, gluing, welding, and the like), wherein the material forming the stiff portion 172 can be stiffer than the material forming the leaflet body 170.

FIG. 19 shows a portion of a leaflet 162^{e} coupled to a strut 114 by stapling member 300. Attachment of leaflet 162^{e} to strut 114 can be performed according to any of the examples described above with respect to FIGS. 10-12, 15A-16 and 31-32B, utilizing any of the exemplary stapling members 300 described above with respect to FIGS. 5A-9B, 13A-14B and 31-33, wherein the stiff portion 172 of the leaflet 162^{e} is positioned adjacent to strut 114, and the legs 318 pierce and extend through the stiff portion 172. Advantageously, the increased stiffness of the stiff portion 172 allows it to better withstand stresses concentrated at the points of leg 318 penetration.

In some examples, the stiff portion width W_{SP} is greater than the strut width W_{S}. In some examples, the stiff portion width W_{SP} is at least 1.5 times greater than the strut width W_{S}. In some examples, the stiff portion width W_{SP} is at least 2 times greater than the strut width W_{S}. In some examples, the stiff portion width W_{SP} is greater than the backspan width W_{B} of backspan 314. In some examples, the stiff portion width W_{SP} is at least 1.5 times greater than the backspan width W_{B}. In some examples, the stiff portion width W_{SP} is at least 2 times greater than the backspan width W_{B}.

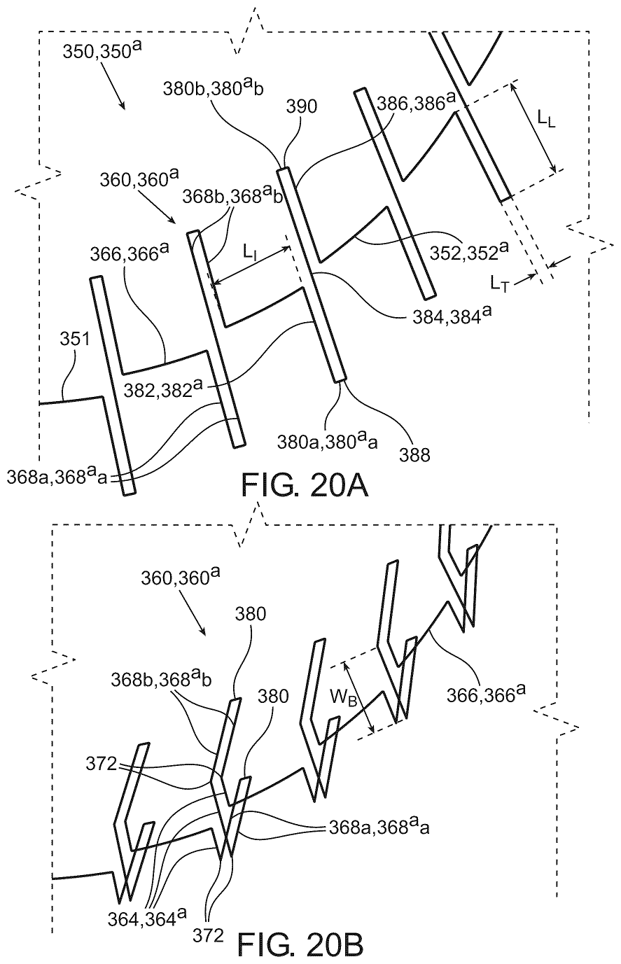

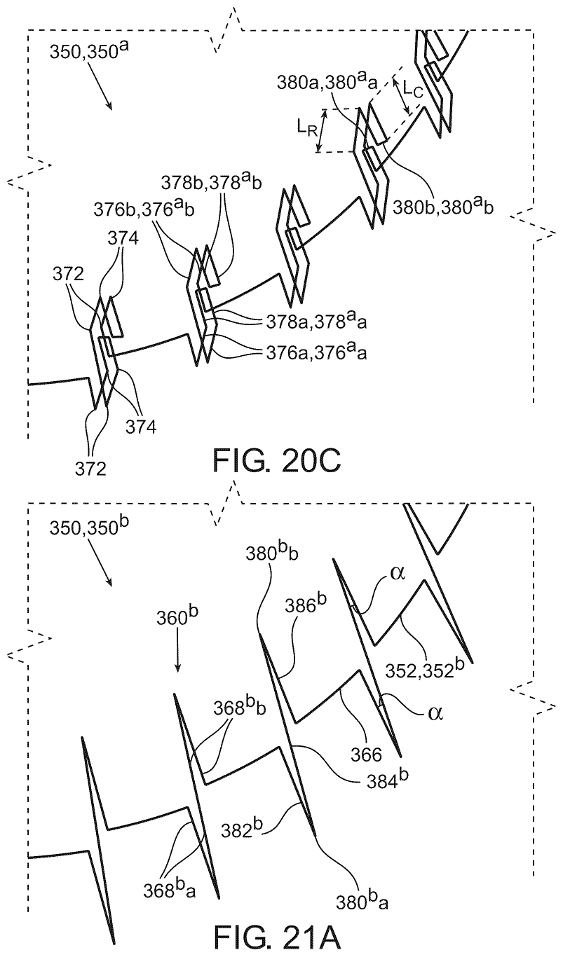

In some examples, a stapling member can be formed from a continuous wire. FIGS. 20A-20C show a portion of an exemplary stapling member 350 that can be used for coupling a leaflet 162 to one or more struts 114 of a frame 110. FIG. 20A shows the stapling member 350 in a flattened configuration. Stapling member 350 is formed from a continuous wire 351 that can be bent to form a spine 352 which can generally extend along stapling member axis Cs in the same manner described above for stapling member 300 and illustrated in FIG. 5A, and a plurality of stapling portions 360 separated from each other by intermediate wire portions 366 of the spine 352. The wire 351 can be made of plastically-deformable materials such as, but not limited to, stainless steel or a nickel based alloy (e.g., a cobalt-chromium or a nickel-cobalt-chromium alloy such as MP35N alloy), titanium, and the like.

The wire 351 of each stapling portion 360 is bent to define two legs 368 extending from the opposite sides of the spine, such as a first leg 368a and a second leg 368^{b}. Each leg 368 terminates at a tip 380, which can be, in some examples, a relatively sharp tip configured to penetrate through the thickness of a leaflet 162, such as through a pericardial tissue the leaflet 162 can be formed of, and in some examples can be a blunt tip that can be passed through pre-formed perforations 176 of a leaflet 1624, in a similar manner to that described above with respect to FIG. 31. Formation of a stapling portion 310 can be achieved by laterally bending the wire 351 from and end of a corresponding intermediate wire portion 366, sideways (relative to the stapling member axis Cs), for example at a right angle (though other angles are contemplated) in a first direction, so as to form a first planar bent extension 382, up to a first tip 380a. The wire 351 is bent to form a second planar bent extension 384 that extends in an opposite direction from the first tip 380a up to a second tip 380^{b}. The wire 351 finally is bent to form a third planar bent extension 386 that extends from the second tip 380^{b} back (i.e., in the first direction) to the starting point of a subsequent intermediate wire portion 366, and is bent, for example at a right angle (though other angles are contemplated) to form the subsequent intermediate wire portion 366. The length of each of the first planar bent extension 382 and third planar bent extension 386 can be similar, is defined as the leg length L_{L} in a similar manner to that described above for a leg length of stapling members 300.

In the flattened configuration shown in FIG. 20A, the first leg 368a can be defined by the first planar bent extension 382 and a portion of the second planar bent extension 384 that extends next to the first planar bent extension 382 along the leg length L_{L}. The second leg 368^{b} can be defined by the third planar bent extension 386 and a portion of the second planar bent extension 384 that extends next to the third planar bent extension 386 along the leg length L_{L}.

In some examples, the tips 380 are formed by relatively linearly lateral bent extensions of the wire 351. FIGS. 20A-20C show an exemplary stapling member 350^{a}, which is an exemplary implementation of stapling member 350, and thus includes all of the features described for stapling member 350 throughout the current disclosure, except that the tips 380^{a} are formed by relatively linearly lateral bent extensions of the wire 351. Specifically, the first tip 380^{a} a is formed by bending the first planar bent extension 382^{a}, for example at a right angle (though other angles are contemplated) to form a first lateral bent extension 388, which is then bent again, for example at a right angle (though other angles are contemplated) to form the second planar bent extension 384^{a}. Similarly, the second tip 380^{a}b is formed by bending the second planar bent extension 384^{a}, for example at a right angle (though other angles are contemplated) to form a second lateral bent extension 390, which is then bent again, for example at a right angle (though other angles are contemplated) to form the third planar bent extension 386^{a}.

The first and second lateral bent extensions 388, 390 can be thin enough or sharpened to allow convenient penetration of the tips 380^{a} through the leaflet material. The lateral tip length L_{T} of each of the first lateral bent extension 388 extending between the first planar bent extension 382^{a} and second planar bent extension 384^{a}, and the second lateral bent extension 390 extending between the second planar bent extension 384^{a} and third planar bent extension 386^{a}, can be substantially less than the leg length L_{L}, to facilitate penetration of the tips through the leaflet material. In some examples, the lateral tip length L_{T} is less than one fifth of the leg length L_{L} (i.e., L_{T}<0.2 L_{L}). In some examples, the lateral tip length L_{T} is less than one tenth of the leg length L_{L} (i.e., L_{T}<0.1 L_{L}).

FIG. 20B shows the stapling member 350 in a first bent configuration, wherein the legs 368 are bent at first bends 372, for example at right angles (though other angles are contemplated) in a radial direction (i.e., out of plane relative to the plane defined by the stapling member 350 in the planar configuration illustrated in FIG. 20A), such that each stapling portion 360 can be generally U-shaped. The portions of the first, second, and third planar bent extensions 382, 384, 386, disposed between the first bends 372 of both legs 368a, 368^{b}, together form the backspan 364 of stapling portion 360, configured to function in a similar manner described above for backspan 314, pressing against the inner surface of the leaflet 162 when coupling the leaflet 162 to a strut 114. The distance between the first bends 372 of both legs 368a, 368^{b} is defined as the backspan width W_{B} in a similar manner to that described above for a backspan width of stapling members 300.

FIG. 20C shows the stapling member 350 in a second bent configuration, wherein the legs 368 are further bent towards each other at second bends 374. As shown, each leg is bent at a second bend 374, between the first bend 372 and the tip 380, dividing the leg 368 into a radial portion 376 extending between the first bend 372 and the second bend 374, and a clinch portion 378 extending between the second bend 374 and the tip 380. The radial portion 376 can extend along a radial portion length L_{R}, and the clinch portion 378 can extend along a clinch portion length L_{C}, in a similar manner to that described above for a radial portion length and a clinch portion length of stapling members 300. Since a portion of the leg 318 also defines part of the backspan 364, up to the first bend 372, the sum of lengths of the remaining radial portion 376 and clinch portion 378 can be less than the leg length L_{L} (i.e., L_{R}+L_{C}<L_{L}).

The first and second clinch portions 378a, 378b extend towards each other, and can be generally parallel to the backspan 364. As shown, the legs 368 of each stapling portion 360 formed in the manner described above, can be offset from each other, such that when the legs 368 are bent at second bends 374, the clinch portion 378 do not intersect with each other but rather extend parallel to each other. As described above with respect to stapling member 3004, this offset configuration allows the clinch portion 378 to be longer than half the backspan width W_{B}, without the risk of the tips 380 contacting or otherwise interfering with each other. This, in turn, can increase surface contact area between the clinch portions 378 and strut 114, to improve clamping force.

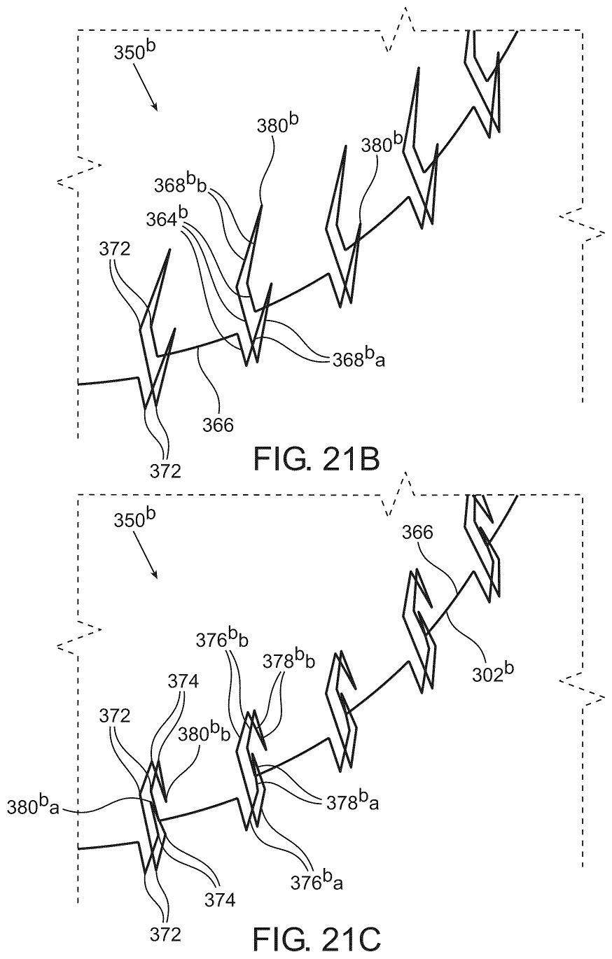

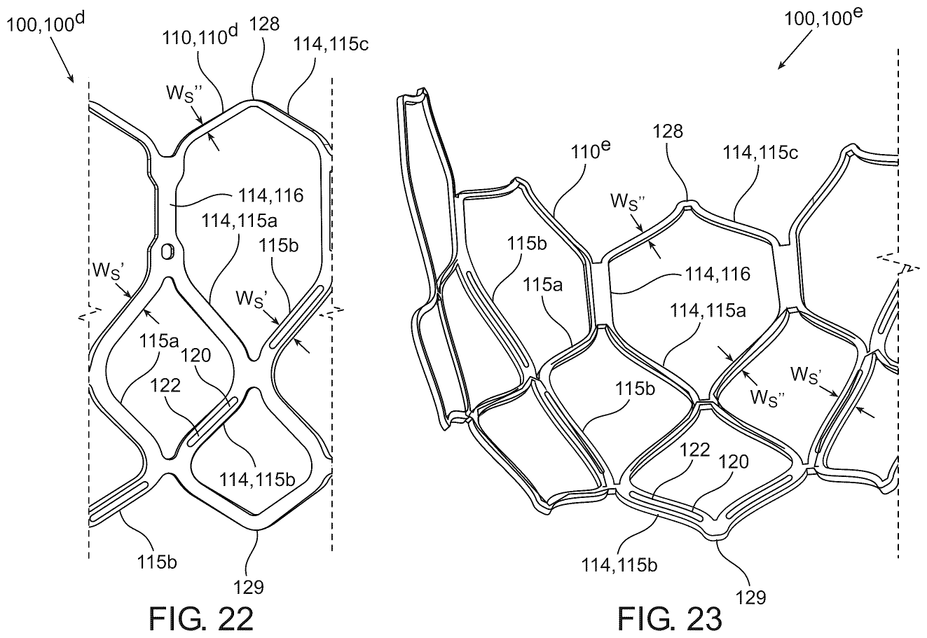

FIGS. 21A-21C show an exemplary stapling member 350^{b}, illustrated in a flattened configuration (FIG. 21A), a first bent configuration (FIG. 22B), and a second bent configuration (FIG. 21C). Stapling member 350^{b} is an exemplary implementation of stapling member 350, and thus includes all of the features described for stapling member 350 throughout the current disclosure, except that the tips 380^{b} are pointed or in the shape of an arrowhead. Each leg 368^{b} can be generally triangular in the flattened configuration shown in FIG. 21A. The first planar bent extension 382^{b} and the second planar bent extension 384^{b} define an angle α therebetween, and the second planar bent extension 384^{b} and the third planar bent extension 386^{b} similarly define the angle α therebetween. In some examples, the angle α is an acute angle. In some examples, the angle α is less than 20°. In some examples, the angle α is less than 10°. In some examples, the angle α is less than 5°.

Utilization of a stapling member 350 for coupling a leaflet 162 to strut 114 can be performed in the same manner described above for stapling members 300 with respect to FIGS. 10-12 and 15A-16, mutatis mutandis. For example, a cusp end portion 165 of a leaflet 162 can be placed adjacent to one or more struts 114 of the frame 110, such that the cusp edge 164 can be somewhat lower than the strut 114, as illustrated in FIG. 10. The leaflet is placed against the inner surface of the frame 110, such that the leaflet 162 is facing the strut inner surface 112. Stapling member 350, in a first bent configuration thereof, can be then approximated to the leaflet 162 from the inner side, such that the legs 368 are oriented toward the leaflet 162. The legs 368 are then pressed into the leaflet 162, causing the tips 380 to pierce through the tissue material, and the legs 368 penetrate into leaflet 162 and extend outward beyond the strut outer surface 113, sandwiching the leaflet 162 between the backspan 364 and the strut 114. Legs 368 are then bent over the outer surface 113 of the strut 114, transitioning the stapling member 350 to the second bent configuration, thus clamping the stapling member 350 over strut 114 to couple the leaflet 162 to the frame 110, for example through and along cusp end portion 165.

In some implementations, the stapling member 350 can be generally curved, meaning that it extends along a curved stapling member axis Cs, optionally following a curvature of the cusp edge 164 and/or cusp end portion 165 of leaflet 162. In some implementation, the curved shape of one or more stapling member 350 can follow a scalloped shape of the scalloped line 105, to allow coupling of the valvular structure 160, by one or more stapling members 300, along a generally scalloped-shaped line 105. Intermediate portion 366 can extend along an intermediate portion length L_{I} (see FIG. 20A) between adjacent stapling portions 360, similar to an intermediate portion length described above with respect to stapling members 300. Different intermediate portions 366 of the spine 352 can have similar or different lengths L_{I}. For example, intermediate portions 366 extending between subsequent stapling portion 310 configured to couple a leaflet 162 to a section of a strut 114 extending between two neighboring junctions 127 can be shorter in length than intermediate portions 316 that extend along junctions 127 of the frame. In some examples, spine 352 can extend in a generally zig-zagged pattern, in a similar manner to that described above with respect to spine 3028 of FIG. 33. While not illustrated explicitly, intermediate wire portions 366 can have offsetting portions angled relative to sub-portions extending from the offsetting portions, similar to offsetting portions 338 and sub-portion 320 described with respect to FIG. 33.

Radial portions 376 of legs 368 extend through the thickness of the leaflet 162 and across the strut 114. In some examples, the radial portion length L_{R} is substantially equal to the combined thicknesses of the leaflet 162 and the strut 114 (i.e., L_{R}≈T_{L}+T_{S}). In some examples, the radial portion length L_{R} is within the range of ±20% of the combined thicknesses of the leaflet 162 and the strut 114, such that 0.8 (T_{L}+T_{S})<L_{R}<1.2 (T_{L}+T_{S}).

As mentioned above, the directions of attachment, extending the legs 368 from the inner side of the frame 110 outwards, may be advantageous, resulting in the relatively sharp tips 380 disposed on the outer side of the valve 100, which reduces the risk of these tips 380 being otherwise engaged by movable portions of the leaflets 162 inside the valve 100.

One or more stapling members 350 can be used to couple leaflets 162 to the struts 114 of a frame 110, in the same manner described for any of the examples above with respect to FIG. 17, mutatis mutandis. For example, any number of stapling members 350 can be utilized. In some examples, a single stapling member 350 can be utilized to couple all leaflets 162 to the frame 110. For example, a single stapling member 350 can have an undulating or scalloped-shaped extending across the complete circumference of the frame 110. In some examples, the number of stapling members 350 can be similar to the number of the leaflets 162, For example, for a tri-leaflet valvular structure 160^{c} shown in FIG. 17, three cusp-shaped stapling members 350 can be used, each formed to generally follow the shape of the cusp of one of the leaflets 162. In some examples, the number of the stapling members 350 can be twice the number of leaflets 162. For example, for a tri-leaflet valvular structure 160^{c} shown in FIG. 17, six cusp-shaped stapling members 350 can be used, each shaped to extend between an inflow apex 129 and a commissure 180, such that each stapling member 350 is used to couple about half of the cusp end portion 165. In some examples, several stapling members 350 can be used to couple different portions of the cusp edge 164 to the frame 110. For example, separate stapling members 350 can be used to extend over each strut 115a (shown in FIG. 17) separately.

The shape of the stapling members 350 can generally follow the shape of the struts 115 to which they are coupled. For example, the second struts 115b illustrated in FIG. 17 are shown to extend in a zig-zagged pattern between each inflow apex 129 and a commissure 180, due to the junctions 127 disposed between adjacent second struts 115b, causing each strut 115b to be offset relative to the neighboring strut 115b. A stapling member 350 configured to extend along more than one strut 115b, can be shaped to exhibit a similar zig-zagged pattern.

In some examples, any of an inner skirt 106 and/or an outer skirt 107 can be added, in which case, the length L_{R} of the radial portions 376 of the legs 368 can be adapted to extend through the thicknesses of the inner skirt 106 and/or outer skirt 107 as well. When an outer skirt 107 is added, the legs 368 can either penetrate through the outer skirt 107 or not. In some examples, the legs 368 extend through the thickness of an outer skirt 107 extending around the frame 110, such that the clinch portions 378 are bent over an outer surface of the outer skirt 107. In some examples, the legs 368 are bent over the struts 115, such that the clinch portions 378 extend over strut outer surfaces 113, and the outer skirt 107 is then attached to the frame 110 around and over the clinch portions 378. Such examples can advantageously conceal the tips 380 from contacting the surrounding anatomy around an implanted prosthetic valve 100.

In some examples, stapling members 350 are used to couple leaflets 162^{e} that includes a stiff portion 172 to the frame 110, in a manner similar to that described above with respect to FIGS. 18-19, mutatis mutandis. In such examples, the legs 368 pierce and extend through the stiff portion 172, wherein the increased stiffness of the stiff portion 172 advantageously allows it to better withstand stresses concentrated at the points of leg 368 penetration. In some examples, the stiff portion width W_{SP} is greater than the backspan width W_{B} of backspan 364. In some examples, the stiff portion width W_{SP} is at least 1.5 times greater than the backspan width W_{B}. In some examples, the stiff portion width W_{SP} is at least 2 times greater than the backspan width W_{B}.

In some examples, compressible inserts can be utilized to clamp leaflets 162 to a frame 110 that includes corresponding recesses into which the compressible inserts can be inserted. FIG. 22 shows a portion of an exemplary frame 110^{d} of a prosthetic valve 100^{d}. Prosthetic valve 100^{d} is an exemplary implementation of prosthetic valve 100, and thus includes all of the features described for prosthetic valve 100 throughout the current disclosure, except that at least some of the struts 114 of frame 110^{d} further include recesses 120 with openings 122 facing the valve central longitudinal axis Ca. In some examples, the frame 110^{d} can comprise a plurality of struts 114, such as angled struts 115 that include, similar to frames 110^{b} and 110^{c} described above, a group of first struts 115a to which the leaflets 162 are not coupled along their cusp end portions 165, and a group of second struts 115b to which the cusp end portions 165 are coupled, forming a scalloped line 105 along selected angled struts 115b.

At least some of the struts 114 of frame 1104, which can be second struts 115b to which leaflets 162 are to be coupled, such as along their cusp end portions 165, include recesses 120. A recess 120, also shown in cross-section in FIG. 25A, includes an opening 122 formed at the strut inner surface 112, and terminates radially inward to strut outer surface 113. Opening 122 defines an opening width W_{O}. While recess 120 is shown to be circular in FIG. 25A, it is to be understood that other shapes, such as elliptical, rectangular, and the like, are contemplated. The recess 120 has a maximal recess width W_{R} in a direction parallel to the opening width W_{O}, and a recess depth H_{R} extending from the opening 122 to an opposite floor of the recess, in a radial direction, parallel to strut thickness T_{S}. In some implementations, when the recess is substantially circular, the maximal recess width W_{R} can be its internal diameter, and the recess depth H_{R} can be optionally also equal to its diameter. The maximal recess width W_{R} is greater than the width W_{O} of opening 122, such that opening 122 extends between two shoulders 124 formed by the strut 114 along the strut inner surface 112. The recess depth H_{R} is smaller than the strut thickness T_{S}.

Struts 114 that include recesses 120 can be formed to have first strut widths Ws′ which are relatively wider than those of equivalent struts 114 of conventional frames 110, devoid of such recesses. Struts 114 that include recesses 120 can be also formed to have first strut thicknesses Ts′ which are relatively thicker than those of equivalent struts 114 of conventional frames 110. In some examples, as illustrated for frame 110^{d} in FIG. 22, angled struts 115b to which the leaflets are coupled, as well as struts 115a to which no leaflets are coupled, can be provided with similar first strut widths Ws′ and/or similar first strut thicknesses Ts′, which are wider and/or thicker, respectively, than those of equivalent struts 114 of conventional frames 110. In some examples, a frame 110^{d} can include a third group of angled struts 115c, such as struts 115 diverging from outflow apices 128. These struts 115c can be disposed at an outflow portion of the frame 110^{d} which is proximal to the valvular structure 160, and more specifically, proximal to the regions of attachment of the leaflets 162, along cusp end portions 165 thereof, to the frame 110^{d}. These struts 115c can be optionally formed with second strut widths Ws″ that can be generally similar to the strut width of conventional frames 110, such that the second strut width Ws″ of outflow struts 115c can be smaller than the first strut width Ws′ of struts 115b that include recesses 120.

FIG. 23 shows a portion of an exemplary frame 110^{e} of a prosthetic valve 100^{e}. Prosthetic valve 100^{e} is an exemplary implementation of prosthetic valve 100^{d}, and thus includes all of the features described for prosthetic valve 100^{d} throughout the current disclosure, except that only the angled struts 115b that include recesses 120 are wider, having the first strut widths Ws′, while other angled struts, such as angled struts 115a, and optionally outflow struts 115c, can remain thinner, having second strut widths Ws″ generally similar to those of equivalent struts 114 of conventional frames 110. In some examples, the first strut width Ws′ is at least 1.5 times greater than the second strut width Ws″. In some examples, the first strut width Ws” is at least 2 times greater than the second strut width Ws″.

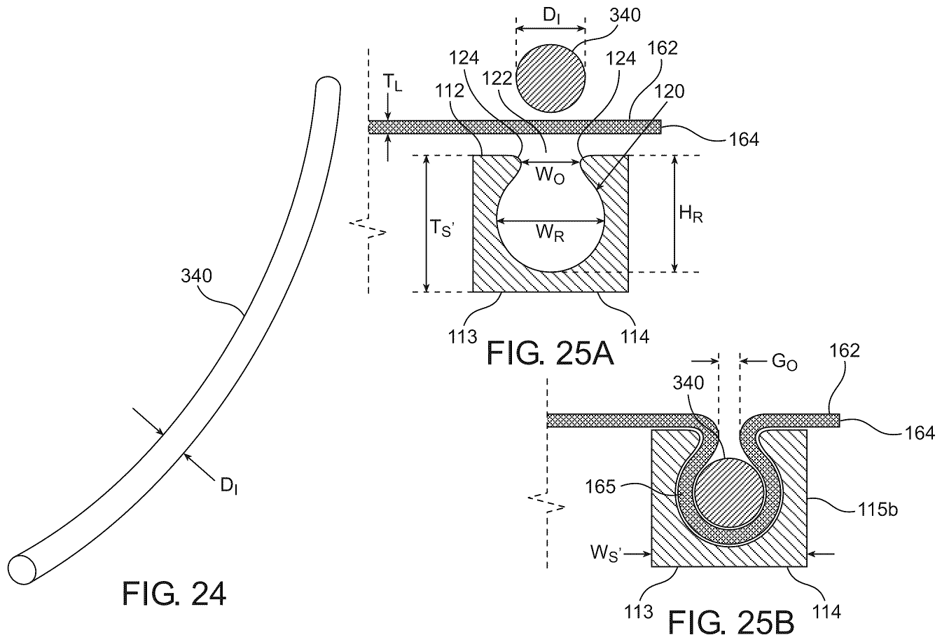

FIG. 24 shows an exemplary compressible insert 340, having an insert diameter D_{I} in a free state thereof. The insert is squeezable to a size which is less than its free state diameter D_{I}, and is configured to be inserted into a corresponding recess 120. Once inside the recess 120, the insert 340 strives to revert back to its free inset diameter D_{I}. The compressible insert 340 can be made from any suitable resilient squeezable material, such as rubber or other polymeric material.

FIGS. 25A-25B shows cross-sectional views of a strut 114 comprising a recess 120, prior to (FIG. 25A) and after (FIG. 25B) coupling a leaflet 162 to the strut 114 with a compressible insert 340. The strut 114 that includes a recess 120 defines a first strut width Ws′ and a first strut thickness Ts′. The maximal recess width W_{R} is smaller than the first strut width Ws′. The recess depth H_{R} is smaller than the first strut thickness Ts′.

As shown in FIG. 25A, a leaflet 162 is approximated to the strut inner surface 112. The compressible insert 340 is pushed from the opposite side of the leaflet 162, so as to push it into the recess 120, as shown in FIG. 25B. In some examples, the insert diameter D_{I} in its free state (shown in FIG. 25A) is greater than the opening width W_{O}, forcing the compressible insert 340 to be squeezed to a diameter which is less than the opening width W_{O} as it is pushed through the opening 122. Since the compressible insert 340 folds the leaflet 162 thereover and drags the folded portion of the leaflet 162 therewith through opening 122, it needs to be able to be squeezed into a width which is less than the opening width W_{O} minus twice the leaflet thickness T_{L}, accounting for the two layers on both sides of the insert 340 dragged therewith through the opening 122. In some examples, the insert diameter D_{I} in its free state does not necessarily needs to be greater than the opening width W_{O}, but rather at least greater than the gap G_{O} between the two layers of leaflet 162 extending through the opening 122 (see FIG. 25B), such that G_{O}=W_{O}−2T_{L}, and D_{I}>Go, but is squeezable to a size of the gap G_{O} when forces to pass through the opening 122.

Once positioned within the recess 120, as shown in FIG. 25B, the insert 340 can expand to a diameter which is greater than the gap G_{O}, which can be its free state diameter D_{I} or a diameter which is less than D_{I} (but still greater than G_{O}), if full expansion of the insert 340 to its free state diameter D_{I} is limited by the folded leaflet layers disposed on both sides of the insert 340, between the insert 340 and the inner walls of the recess 120. As further shown in FIG. 25B, the leaflet in this final configuration extends around both shoulders 124, wherein the insert 340 disposed within the recess 120 is pressing the leaflet portions against the shoulders 124, such that the shoulders 124 serve as ridges that prevent the leaflet 162 and insert 340 from spontaneously sliding out of the recess 120. The effectively snap-fits the leaflet 162 into recess 120. In some examples, the insert diameter D_{I} is smaller than the recess depth H_{R}. In some examples, the sum of insert diameter D_{I} and twice the leaflet thickness T_{L} is smaller than the recess depth H_{R}, such that D_{I}<(H_{R}-2T_{L}).

In some examples, a plurality of struts 114, such as the plurality of angled second struts 115b, can include a corresponding plurality of recesses 120. For example, one recess 120 can extend between the junctions 127 (or a junction 127 and an inflow apex 129) of each corresponding strut 114 to which the leaflet 162 is to be coupled. As shown in FIGS. 22-23. The struts 115b that include recesses 120 can be struts that generally follow a scalloped-shaped line. In some examples, the length of the plurality of recesses 120 can be non-equal, for example, when formed into struts 114 having non-equal lengths. While each strut 115b is shown in FIGS. 22-23 to include a single recess, it is to be understood that in some example, more than one recess 120 can be formed in a single strut 114. While adjacent recesses 120 are shown in FIGS. 22-23 to be separated from each other by junctions 127, it is to be understood that in some examples, a single recess can span across more than one strut 114, formed in a junction 127 between the struts 114 as well.

The length of the compressible insert 340 can be similar to or less than the length of the corresponding recess 120. In some examples, a single compressible insert 340 is inserted into a corresponding one of the recesses 120 to couple the leaflet 162 to the strut 114. In some examples, a plurality of compressible insert 340, each of which is shorter in length than the corresponding recesses 120, are inserted into the corresponding recess 120 to couple the leaflet 162 to the strut 114.

Strut 114 that includes a recess, are also referred to as “recessed struts”. Thus, the terms “recessed struts” and “second struts 115b”, for example with reference to prosthetic valves such as 100^{d} or 100^{e}, are interchangeable. Similarly, struts 114 which are devoid of recesses 120, are also referred to as “unrecessed struts”. Thus, the term “unrecessed struts” can be used interchangeably with any of “first struts 115a” and/or “third struts 115c”, for example with reference to prosthetic valves such as 100^{d} or 100^{e}. The cross-sectional view of FIGS. 25A-25B are taken across recessed struts. While angled struts 115b are described above and illustrated in FIGS. 22-23 as recessed struts, it is to be understood that this is not meant to be limiting, and that recessed struts can similarly include axial struts. While leaflets 162 are described above to be coupled to recessed struts along cusp end portions 165 thereof, this is not meant to be limiting, and other portions of the leaflets, such as those including tabs or equivalent regions extending between the cusp edges 164 and the free edges 166 can be also coupled to recesses formed, for example, in vertical struts, thereby coupling the leaflets to the frame at commissure regions as well.

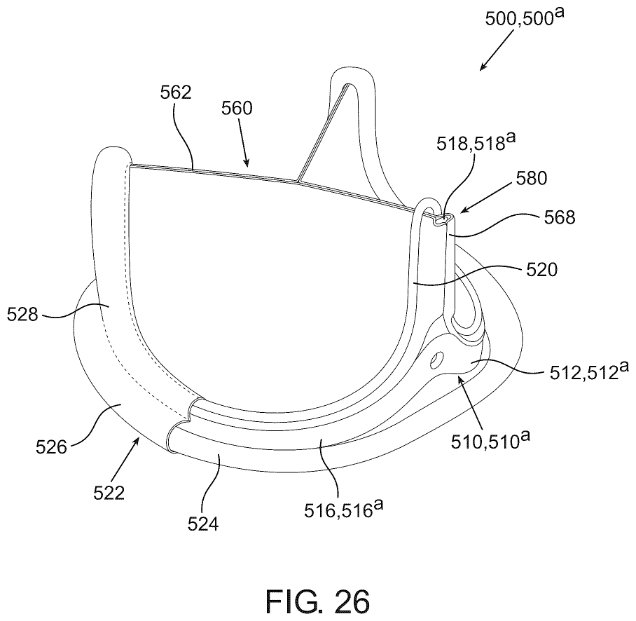

FIG. 26 shows an example of a conventional surgically implantable prosthetic valve 500. In the particular illustrated example, the prosthetic valve 500 comprises a support frame 510 and a valvular structure 560 attached thereto. The valvular structure 560 comprises a plurality of leaflets 562 (e.g., three leaflets), positioned at least partially within the frame 510, and configured to regulate flow of blood through the prosthetic valve 500. While three leaflets 562 arranged to collapse in a tricuspid arrangement similar to the native aortic valve, are shown in the example illustrated in FIG. 26, it will be clear that a prosthetic valve 500 can include any other number of leaflets 562, such as two leaflets configured to collapse in a bicuspid arrangement similar to the native mitral valve, or more than three leaflets, depending upon the particular application. The leaflets 562 can be generally similar to leaflets 162 described above, and can be made of a flexible material, derived from biological materials (e.g., bovine pericardium or pericardium from other sources), bio-compatible synthetic materials, or other suitable materials as known in the art and described, for example, in U.S. Pat. Nos. 6,730,118, 6,767,362 and 6,908,481, which are incorporated by reference herein.

The frame 510 comprises base portion 512 which is generally rigid and/or expansion-resistant in order to maintain the particular shape and diameter of the prosthetic valve 500, and a plurality of vertically oriented commissure posts 518 (e.g., three posts) extending proximally from the base portion 512 to support the free edges 566 of the leaflets 562. The base portion 512 can comprise cusp portions 516 extending between the vertically oriented commissure posts 518. The frame 510 can be metallic, plastic, or a combination of the two.

Each of the leaflets 562 can be attached along a cusp edge 564 thereof to a corresponding cusp portion 516 of the frame 510 and up along adjacent commissure posts 518. Each leaflet 562 can include a pair of oppositely-directed tabs 568, wherein each respective tab 568 can be aligned with a tab 568 of an adjacent leaflet 562 as shown. The tabs 568 can be wrapped around a respective commissure post 518 of the frame 510 and coupled thereto, thereby forming commissures 580 that project in an outflow direction along the valve central longitudinal axis Ca.

A soft sealing or sewing ring 522 circumscribes the frame 510, for example around the base portion 512, and is typically used to secure the prosthetic valve to a native annulus such as with sutures. The sewing ring 522 comprises a sewing ring insert 524 and a cloth cover 526. The sewing ring insert 524 can be made of a suture permeable material for suturing the prosthetic valve to a native annulus, as known in the art. For example, the sewing ring insert 524 can be made of a silicone-based material, although other suture-permeable materials can be used. The cloth cover 526 can be formed of any biocompatible fabric, such as, for example, polyethylene terephthalate or polyester fabric.

A skirt 528 can completely cover the frame 510, including the base portion 512 and the commissure posts 518. The sewing ring 522 can be secured to the frame 510 by being stitched to the skirt 528, or via sutures extending through the sewing ring 522, and optionally apertures of the base portion 512, when the base portion 512 comprises such apertures. The skirt 528 can be formed of any biocompatible fabric, such as, for example, polyethylene terephthalate or polyester fabric.

FIG. 26 illustrates one example of a conventional surgically implantable prosthetic valve 500^{a}. Prosthetic valve 500^{a} is an exemplary implementation of prosthetic valve 500, and thus includes all of the features described for prosthetic valve 500 above, except that the leaflets 562 are coupled to the base portion 512^{a} of the frame 510^{a} by sutures. In some configurations, as illustrated, the surgically implantable prosthetic valve 500^{a} further comprises an undulating wireform 520, configured to provide further support to the leaflets 562. The wireform 520 can include a plurality (e.g., three) large radius wireform cusps supporting the cusp regions of the valvular structure 560, while the ends of each pair of adjacent wireform cusps converge somewhat asymptotically to form upstanding wireform commissure portions that terminate in tips, each extending in the opposite direction as the arcuate wireform cusps and having a relatively smaller radius. The cusp portions 516^{a} and the commissure posts 518^{a} can be sized and shaped so as to correspond to the curvature of the wireform 520.

Wireform 520 typically is formed from one or more pieces of wire but also can be formed from other similarly-shaped elongate members. The wireform can also be cut or otherwise formed from tubing or a sheet of material. The wireform can have any of various cross sectional shapes, such as a square, rectangular, circular, or combinations thereof. In some implementations, wireform 520 is made of a relatively rigid metal, such as stainless steel or Elgiloy (a Co—Cr—Ni alloy). In some implementations, the wireform 520 further comprises a wireform cloth encapsulating it along its length.

In some implementations, the base portion 512^{a} can be in the form of a band, the leaflets 562 can be sutured along cusp edges 564 thereof to corresponding cusp portion 516^{a} of the base portion 512^{a} and extend up along adjacent commissure posts 518^{a}. Each pair of aligned tabs 568 can be inserted between adjacent upstanding wireform portions. The tabs 568 can then be wrapped around a respective commissure posts 518^{a} of the frame 510^{a}, and sutured to each other and/or to the commissure post 518^{a} to form commissures 580. The wireform 520 and the commissure posts 518^{a} can provide flexibility to the commissures 580 which helps reduce stress on the bioprosthetic material of the leaflets 562. The wireform 520 is secured to the inner side of the frame 510^{a}, wherein the skirt 528 can, in some examples, cover the wireform 520 as well.

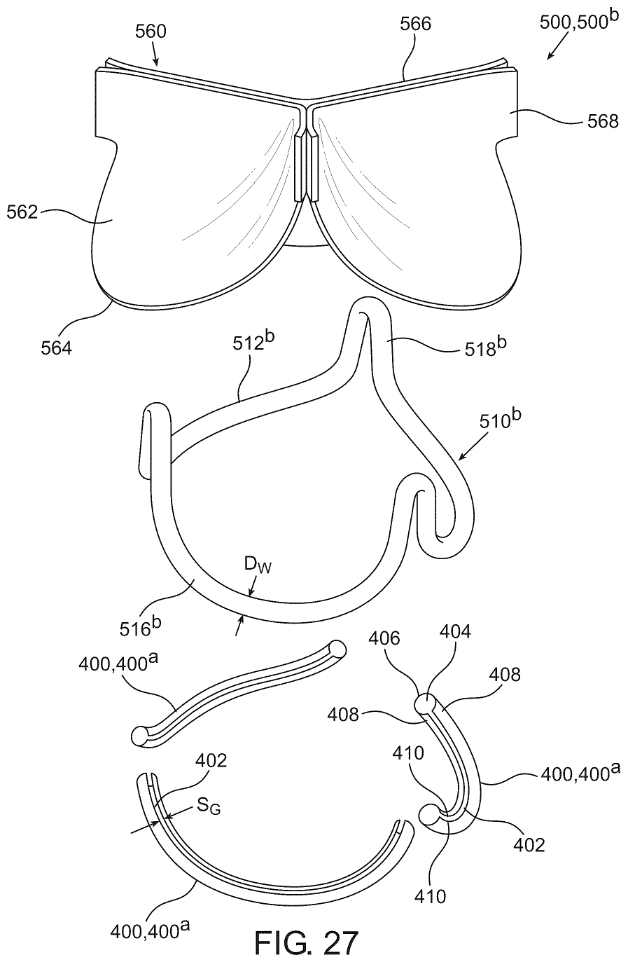

FIGS. 27-29B show an exemplary surgically implantable prosthetic valve 500^{b}. Prosthetic valve 500^{b} is an exemplary implementation of prosthetic valve 500, and thus includes all of the features described for prosthetic valve 500 above, except that the leaflets 562b are coupled to the base portion 512b of the frame 510^{b} by one or more gripping tubes 400. FIG. 27 is an exploded view of components of prosthetic valve 500^{b}, including valvular structure 560, frame 510^{b} and gripping tubes 400, prior to attachment to each other. In some implementations, the frame 510^{b} can be formed as a wire or tubular member, having a circular or elliptical cross-sectional shape, though other cross-sectional shapes of the frame 510^{b} are contemplated. When formed with a circular cross-sectional shape, the frame 510^{b}, or at least cusp portion 516^{b} thereof, can define a frame tubular diameter D_{W}. If the frame 510^{b} is not circular, its width, or the width of cusp portion 516^{b} thereof, can be equal to D_{W}.

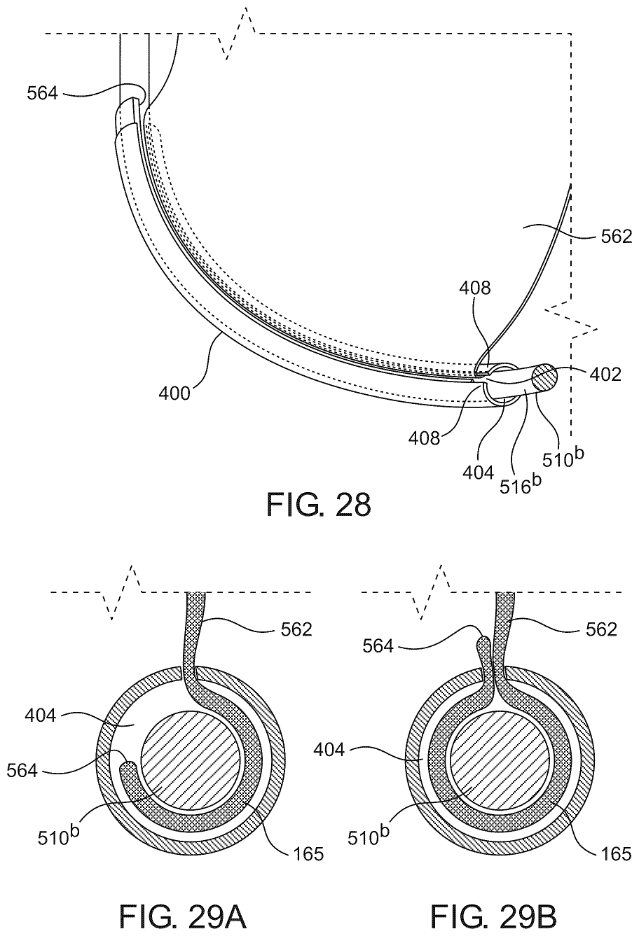

FIG. 28 shows a portion of assembled prosthetic valve 500^{b}, with a gripping tube 400 clamped around a cusp portion 516^{b} with a portion of leaflet 562 wrapped around the cusp portion 516^{b} inside an inner channel 404 defined by the gripping tube 400. Gripping tube 400 is configured to couple a leaflet 562 to a cusp portion 516^{b} of frame 510^{b} by clamping over a portion of the leaflet 562, for example along the cusp edge 564, wrapped around a corresponding cusp portion 516^{b}, such that the gripping tube 400 snap-fits or clips over the wrapped cusp portion 516^{b}.

Gripping tube 400 comprises a mid-portion 406 and a couple of side arms 408 biased towards each other, the side arms 408 extending in a continuous manner from both sides of the mid-portion 406 and terminating at free ends 410. As illustrated, the gripping tube 400 includes a slot 402, and can be C-shaped in cross-section with the side arms 408 arched inward and terminating at free ends 410 on both sides of the slot 402, such that the slot 402 defines a gap between the free ends 410, through which a portion of the frame 510^{b}, such as cusp portion 516^{b} or any other part of the base portion 512b, optionally wrapped by a portion of leaflet 562, may pass toward the mid-portion 406. In some implementation, the gripping tube 400 if formed as a single monolithic component, for example formed from a single tube, along which a slit is cut to form the slot 402. In alternative implementations, the gripping tube 400 may be comprised of more than one component, for example by having two separately formed side arms that may be affixed to two opposing edges of a mid-portion. The slot 402 defines a slot width S_{G} in a free state of the gripping tube 400. In some examples, the cross-sectional shape of the inner channel 404 of gripping tube 400 is similar, yet greater in size, than the cross-sectional shape of frame 510^{b} and/or cusp portions 516^{b} thereof.

The side arms 408 are resiliently expandable away from each other, such that a cusp portion 516^{b}, optionally wrapped, fully or partially, by leaflet 562, is passable through the slot 402 formed between their free ends 410. In use, a gripping tube 400 can be pushed over a cusp portion 516^{b} which is wrapped fully or partially by leaflet 562. The slot width S_{G} can be smaller than the frame tubular diameter D_{W}. When the gripping tube 400 is pushed over the wrapped a cusp portion 516^{b}, the cusp portion 516^{b}, optionally with the portion of the leaflet 562 wrapped therearound, may apply a force sufficient to expand the side arms 408 away from each other, so as to allow the wrapped a cusp portion 516^{b} to pass through the slot 402 into inner channel 404, toward the mid-portion 406. Once the cusp portion 516^{b} is completely accommodated within inner channel 404, and in the absence of further expanding force applied to the side arms 408, the side arms resiliently snap back toward each other to compress against the portion of the leaflet 562 extending from the cusp portion 516^{b} through the slot 402, in order to clamp the leaflet 562 around the cusp portion 516^{b} of frame 510^{b}.

FIG. 29A shows a cross-sectional view of an optional configuration in which the leaflet 562 is partially wrapped around the frame 510^{b}, such as around a cusp portion 516^{b}. In this configuration, the leaflet 562 is wrapped around a part of the circumference of frame 510^{b}, with the cusp edge 564 positioned within the inner channel 404, such that only a single layer of leaflet 562 extends through slot 402. In some examples, the gap width S_{G}, in a free state of the gripping tube 400, is smaller than the leaflet thickness T_{L}, such that the side arms 408 snap towards each other against the portion of the leaflet 562 extending through the slot 402. In some cases, the leaflet material, such as pericardium, is squeezable, for example up to half of its free-state thickness, such that the side arms 408 can pinch the leaflet 562 and optionally retain it in a somewhat squeezed thickness within the slot 402, with both of the free end 410 pressing against both sides of the leaflet.

FIG. 29B shows a cross-sectional view of an optional configuration in which the leaflet 562 is partially wrapped around the frame 510^{b}, such as around a cusp portion 516^{b}. In this configuration, the leaflet 562 extends into the inner channel 404 through the gap of slot 402, circumscribes the frame 510^{b}, and extends back through the slot 402 out of the inner channel 404, such that the cusp edge 564 is disposed out of the inner channel 404, resulting in two layers of the leaflet 562 extending through slot 402. The side arms 408 in this configurations can snap towards each other against the portions of the leaflet 562 extending into and out of the slot 402. In some examples, the slot width S_{G}, in a free state of the gripping tube 400, is smaller than twice the leaflet thickness T_{L} (i.e., S_{G}<2T_{L}), such that the side arms 408 can pinch both layers of the leaflet 562 extending through the slot 402 against each other, with one of the free ends 410 pressing against one layer of the leaflet 562 extending through the slot 402 into the inner channel, and the other free end 410 pressing against the layer of the leaflet 562 extending through the slot 402 out of the inner channel 404.

The gripping tube 400 defines an inner channel diameter D_{IC}. In some examples, the inner channel diameter D_{IC} is larger than the frame tubular diameter D_{W}. In some examples, the inner channel diameter D_{IC} is larger than the sum of the frame tubular diameter D_{W} and the thickness of one layer of the leaflet (i.e., D_{IC}>(D_{W}+T_{L})). In some examples, the inner channel diameter D_{IC} is equal to or smaller than the sum of the frame tubular diameter D_{W} and twice the thickness the leaflet (i.e., D_{IC}≤(D_{W}+2T_{L})). In some examples, the inner channel diameter D_{IC} is smaller than the sum of the frame tubular diameter D_{W} and three times the thickness the leaflet (i.e., D_{IC}< (D_{W}+3T_{L})).

While not explicitly shown for prosthetic valve 500^{b}, it is to be understood that additional components, such as a sewing ring 522 and/or a skirt 528, cab be added after coupling the valvular structure 560 to the frame 510^{b}, in a similar manner to that described above for surgically implantable prosthetic valve 500.

In some examples, a plurality of gripping tubes 400 are used to couple a valvular structure 560 to the frame 510^{b}. FIG. 27 shows a plurality of exemplary gripping tubes 400^{a}, and more specifically, three gripping tubes 400^{a}, corresponding to the three cusp portions 516^{b} of the frame 510^{b}. Each gripping tube 400^{a} can have a curved shape generally mimicking the curved shape of the corresponding cusp portion 516^{b}, and having a length that extends along whole or part of the length of the corresponding cusp portion 516^{b}. While the number of gripping tubes 400^{a} illustrated in FIG. 27 matches the number of cusp portion 516^{b}, it is to be understood that in some examples, a plurality of relatively shorter gripping tubes 400 can be clamped over a single corresponding cusp portion 516^{b}.

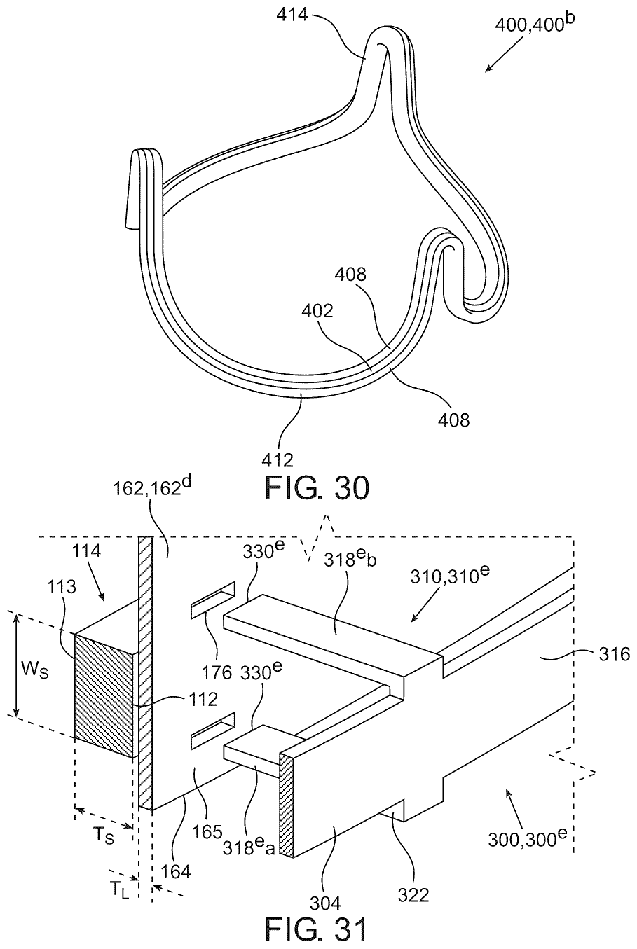

In some examples, one or more gripping tubes 400 can be similarly utilized to couple leaflets 562 not only to the cusp portion 516^{b}, but rather to the vertically oriented commissure posts 518^{b} of frame 510^{b} as well. FIG. 30 shows an exemplary gripping tube 400^{b}, which can be a single unit utilized to clamp the entire valvular structure 560 to frame 510^{b}. The shape of the gripping tube 400^{b} can generally mimic the shape of the entire frame 510^{b}. The gripping tube 400^{b} can include gripping tube cusp portions 412, following the shape of the cusp portions 516^{b} of the frame 510^{b}, and gripping tube commissure portions 414, corresponding to the U-shaped vertically oriented commissure posts 518^{b} of the frame 510^{b}. In such cases, the leaflet 562 may be provided without tabs 568, but rather shaped to include a continuous edge that can be wrapped around the entire frame 510^{b}, and clamped thereto by the gripping tube 400^{b}.

In some examples, a plurality of differently shaped gripping tubes 400 can be utilized to clamp the valvular structure 560 to different portions of the frame 510^{b}. For example, a plurality (e.g., three) gripping tubes 400, shaped in a similar manner to gripping tubes 400^{a}, can be used to couple the leaflets 562 to the cusp portions 516^{b} of the frame 510^{b}, and a separate plurality (e.g., three) of gripping tubes 400, which can be shaped in a similar manner to U-shaped gripping tube commissure portions 414 of gripping tube 400^{b}, can be utilized to couple the leaflets 562 to the vertically oriented commissure posts 518^{b} of the frame 510^{b}.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising:
a frame movable between a radially compressed state and a radially expanded state;
a valvular structure coupled to the frame and comprising a plurality of leaflets configured to regulate flow through the prosthetic valve; and
at least one stapling member coupling at least one of the plurality of leaflets to the frame, the at least one stapling member comprising:
   a spine extending along a stapling member axis; and
   a plurality of stapling portions separated from each other by intermediate portions of the spine, wherein each stapling portion comprises at least one leg extending from a backspan;
wherein the at least one leg of each stapling portion extends through a corresponding one of the plurality of leaflets, and is bent around a corresponding strut of a plurality of struts of the frame, so as to couple the leaflet to the frame.

Example 2. The prosthetic valve of any example herein, particularly example 1, wherein each leaflet comprises:
a cusp edge;
a free edge opposite to the cusp edge;
a cusp end portion extending from the cusp edge; and
a leaflet body extending from the cusp edge to the free edge;
wherein the at least one leg extending through each of the plurality of leaflets, extends through the cusp end portion of the leaflet.

Example 3. The prosthetic valve of any example herein, particularly example 1 or 2, wherein the backspan is perpendicular to the stapling member axis.

Example 4. The prosthetic valve of any example herein, particularly example 1 to 3, wherein the at least one leg of each stapling portion is angled relative to the stapling member axis.

Example 5. The prosthetic valve of any example herein, particularly example 4, wherein the at least one leg of each stapling portion is orthogonal to the stapling member axis.

Example 6. The prosthetic valve of any example herein, particularly any one of examples 1 to 5, wherein the at least one leg terminates at a tip configured to pierce through the corresponding leaflet.

Example 7. The prosthetic valve of any example herein, particularly example 6, wherein the tip comprises a slanted surface.

Example 8. The prosthetic valve of any example herein, particularly example 6, wherein the tip is arrow-shaped.

Example 9. The prosthetic valve of any example herein, particularly example 6, wherein the tip is conical.

Example 10. The prosthetic valve of any example herein, particularly any one of examples 1 to 5, wherein each leaflet comprises at least one pre-formed aperture through which a corresponding one of the at least one legs extends.

Example 11. The prosthetic valve of any example herein, particularly example 10, wherein the at least one leg terminates at a blunt tip.

Example 12. The prosthetic valve of any example herein, particularly any one of examples 1 to 11, wherein the leaflet is sandwiched between the spine and the frame.

Example 13. The prosthetic valve of any example herein, particularly example 2, wherein the cusp end portion is pressed between the backspan and the strut.

Example 14. The prosthetic valve of any example herein, particularly any one of examples 1 to 13, wherein the spine defines a flat spine outer surface which is pressed against the leaflet coupled thereby to the frame.

Example 15. The prosthetic valve of any example herein, particularly any one of examples 1 to 14, wherein the spine and the stapling portions are integrally formed.

Example 16. The prosthetic valve of any example herein, particularly any one of examples 1 to 14, wherein the stapling portion comprises a stapling element provided as a separately formed component which is attached to the spine, wherein the stapling element comprises the backspan and the at least one leg.

Example 17. The prosthetic valve of any example herein, particularly example 16, wherein the backspan is attached to the spine.

Example 18. The prosthetic valve of any example herein, particularly any one of examples 1 to 17, wherein each leg of the stapling portion is bent at a first bend relative to the backspan.

Example 19. The prosthetic valve of any example herein, particularly example 18, wherein the first bend defines a right angle between the backspan and the corresponding leg.

Example 20. The prosthetic valve of any example herein, particularly example 18 or 19, wherein the at least one leg is bent at a second bend that divides the leg to a radial portion extending between the first bend and the second bend, and a clinch portion extending from the second bend.

Example 21. The prosthetic valve of any example herein, particularly example 20, wherein the second bend defines a right angle between the radial portion and the clinch portion.

Example 22. The prosthetic valve of any example herein, particularly any one of examples 20 to 21, wherein the radial portion of each stapling portion extends radially through the corresponding leaflet and across the corresponding strut the leaflet is coupled to.

Example 23. The prosthetic valve of any example herein, particularly any one of examples 20 to 22, wherein the radial portion defines a radial portion length which is at least as great as the sum of thicknesses of the leaflet and the strut.

Example 24. The prosthetic valve of any example herein, particularly any one of examples 20 to 23, wherein the at least one leg is bent at a third bend that divides the clinch portion to an axial clinch section extending between the second bend and the third bend, and a radial clinch section extending from the third bend.

Example 25. The prosthetic valve of any example herein, particularly example 24, wherein the radial clinch section is parallel to the radial portion.

Example 26 The prosthetic valve of any example herein, particularly example 24 or 25, wherein the leg defines a leg length, wherein the strut defines a strut thickness and a strut width, and wherein the leg length is greater than the sum of the strut width and the strut thickness.

Example 27. The prosthetic valve of any example herein, particularly example 26, wherein the leg length is smaller than the sum of the strut width and twice the strut thickness.

Example 28. The prosthetic valve of any example herein, particularly any one of examples 1 to 27, wherein the at least one leg of the stapling portion comprises a single leg.

Example 29. The prosthetic valve of any example herein, particularly any one of examples 6 to 9 or 11, wherein the at least one leg of the stapling portion comprises a pair of legs, and wherein the tips of the pair of legs are oriented towards each other.

Example 30. The prosthetic valve of any example herein, particularly any one of examples 18 to 23, wherein the at least one leg of the stapling portion comprises a pair of legs extending from opposite ends of the backspan.

Example 31. The prosthetic valve of any example herein, particularly example 30, and wherein the clinch portions of the legs of the stapling portion are oriented towards each other.

Example 32. The prosthetic valve of any example herein, particularly any one of examples 30 to 31, wherein the legs of the stapling portion are aligned with each other.

Example 33. The prosthetic valve of any example herein, particularly example 33, wherein the clinch portions of the stapling portion are coaxial.

Example 34. The prosthetic valve of any example herein, particularly example 32 or 33, wherein each clinch portion defines a clinch portion length, and wherein the backspan defines a backspan width, such that the backspan width is at least two times greater the clinch portion length.

Example 35. The prosthetic valve of any example herein, particularly example 32 or 33, wherein each clinch portion defines a clinch portion length, and wherein the strut defines a strut width, such that the strut width is at least two times greater the clinch portion length.

Example 36. The prosthetic valve of any example herein, particularly example 30, wherein the legs of the stapling portion are offset from each other.

Example 37. The prosthetic valve of any example herein, particularly example 36, wherein the clinch portions of the stapling portion are parallel to each other.

Example 38. The prosthetic valve of any example herein, particularly example 36 or 37, wherein each clinch portion defines a clinch portion length, and wherein the backspan defines a backspan width, such that the clinch portion length is greater than half the backspan width.

Example 39. The prosthetic valve of any example herein, particularly example 36 or 37, wherein each clinch portion defines a clinch portion length, and wherein the strut defines a strut width, such that the clinch portion length is greater than half the strut width.

Example 40. The prosthetic valve of any example herein, particularly any one of examples 29 to 39, wherein one leg of the pair of legs is shorter than the other leg of the pair of legs.

Example 41. The prosthetic valve of any example herein, particularly any one of examples 20 to 40, wherein the clinch portion extends over an outer surface of the strut.

Example 42. The prosthetic valve of any example herein, particularly any one of examples 1 to 41, wherein the stapling member is formed from a plate cut to form a planar configuration of the stapling member prior to being used for coupling the leaflet to the frame, and bent to form a second bent configuration of the stapling member when used to couple the leaflet to the frame.

Example 43. The prosthetic valve of any example herein, particularly any one of examples 1 to 41, wherein the stapling member is formed from a wire, and wherein the intermediate portions are intermediate wire portions.

Example 44. The prosthetic valve of any example herein, particularly example 43,
wherein the wire is bent to form the plurality of stapling portions in a planar configuration of the stapling member prior to being used for coupling the leaflet to the frame, wherein each stapling portion comprises, in the planar configuration of the stapling member:
   a first planar bent extension, terminating at a first tip;
   a second planar bent extension, extending from the first tip to an opposite second tip; and
   a third planar bent extension extending from the second tip.

Example 45. The prosthetic valve of any example herein, particularly example 44, wherein the first tip comprises a first lateral bent extension between the first planar bent extension and the second planar bent extension, and wherein the second tip comprises a second lateral bent extension between the second planar bent extension and the third planar bent extension.

Example 46. The prosthetic valve of any example herein, particularly example 44, wherein the first tip defines an acute angle between the first planar bent extension and the second planar bent extension, and wherein the second tip defines a sharp angle between the second planar bent extension and the third planar bent extension.

Example 47. The prosthetic valve of any example herein, particularly any one of examples 1 to 46, wherein each cusp end portion comprises a stiff portion which is stiffer than a leaflet body of the leaflet.

Example 48. The prosthetic valve of any example herein, particularly example 47, wherein the stiff portion defines a stiff portion width between a cusp edge of the leaflet and a stiff portion proximal end, such that a leaflet body extends between the stiff portion proximal end and a free edge of the leaflet.

Example 49. The prosthetic valve of any example herein, particularly example 48, wherein the stiff portion width is at least 1.5 times greater than a strut width defined by the strut to which the leaflet is coupled by the stapling member.

Example 50. The prosthetic valve of any example herein, particularly example 49, wherein the stiff portion width is at least 2 times greater than a strut width.

Example 51. The prosthetic valve of any example herein, particularly any one of examples 48 to 50, wherein the stiff portion has an ultimate tensile stress which is at least 1.5 times greater than an ultimate tensile stress of the leaflet body.

Example 52. The prosthetic valve of any example herein, particularly example 51, wherein the ultimate tensile stress of the stiff portion is at least 2 times greater than the ultimate tensile stress of the leaflet body.

Example 53. The prosthetic valve of any example herein, particularly any one of examples 48 to 52, wherein the stiff portion obtains a load at failure which is at least 2 times greater than a load at failure obtained by the leaflet body.

Example 54. The prosthetic valve of any example herein, particularly any one of examples 48 to 53, wherein the stiff portion is coated by a reinforcing layer.

Example 55. The prosthetic valve of any example herein, particularly any one of examples 1 to 54, wherein the stapling member axis is curved.

Example 56. The prosthetic valve of any example herein, particularly any one of examples 1 to 55, wherein the at least one stapling member comprises a plurality of stapling members, together forming a scalloped-shaped line along which the valvular structure is coupled to the frame by the stapling members.

Example 57. The prosthetic valve of any example herein, particularly any one of examples 1 to 54, wherein the spine is zig-zagged.

Example 58. The prosthetic valve of any example herein, particularly any one of examples 1 to 54, wherein the spine comprises at least one offsetting portions.

Example 59. The prosthetic valve of any example herein, particularly example 58, wherein the at least one offsetting portion is aligned with at least one junction of the frame.

Example 60. The prosthetic valve of any example herein, particularly any one of examples 1 to 59, wherein the plurality of leaflets comprises three leaflets.

Example 61. The prosthetic valve of any example herein, particularly any one of examples 1 to 60, wherein the stapling member is formed of a plastically deformable material.

Example 62. The prosthetic valve of any example herein, particularly any one of examples 1 to 61, further comprising an inner skirt coupled to the frame.

Example 63. The prosthetic valve of any example herein, particularly example 62, wherein the legs extend through the inner skirt.

Example 64. The prosthetic valve of any example herein, particularly any one of examples 1 to 63, further comprising an inner skirt coupled to the frame.

Example 65. A method of assembling a prosthetic valve, comprising:
providing a stapling member in a first bent configuration, comprising a spine extending along a stapling member axis and a plurality of U-shaped stapling portions separated from each other by intermediate portions of the spine, each stapling portion comprising a backspan and a pair of legs bent relative to the backspan at first bends of the legs;
approximating a leaflet to a strut of a frame of a prosthetic valve;
pushing the legs toward the leaflet such that tips of the legs pierce through the leaflet;
extending the legs radially through the leaflet and across the strut; and
transitioning the stapling member to a second bent configuration by bending the legs at second bends thereof over the struts, so as to couple the leaflet to the strut.

Example 66. The method of any example herein, particularly example 65, wherein the legs are parallel to each other at the first bent configuration.

Example 67. The method of any example herein, particularly example 65 or 66, wherein approximating a leaflet to the strut comprises approximating a cusp end portion of the leaflet to the strut.

Example 68. The method of any example herein, particularly example 67, wherein pushing the legs toward the leaflet comprising pushing the legs toward the cusp end portion such that tips of the legs pierce through the cusp end portion.

Example 69. The method of any example herein, particularly example 67 or 68, wherein extending the legs radially through the leaflet comprising extending the legs radially through the cusp end portion.

Example 70. The method of any example herein, particularly example 65 or 66, wherein approximating a leaflet to the strut comprises approximating a stiff portion of the leaflet to the strut, and wherein the leaflet comprises a leaflet body extending between the stiff portion and a free edge of the leaflet.

Example 71. The method of any example herein, particularly example 70, wherein pushing the legs toward the leaflet comprising pushing the legs toward the stiff portion such that tips of the legs pierce through the stiff portion.

Example 72. The method of any example herein, particularly example 70 or 71, wherein extending the legs radially through the leaflet comprising extending the legs radially through the stiff portion.

Example 73. The method of any example herein, particularly any one of examples 70 to 72, wherein the stiff portion has an ultimate tensile stress which is at least 1.5 times greater than an ultimate tensile stress of the leaflet body.

Example 74. The method of any example herein, particularly example 73, wherein the ultimate tensile stress of the stiff portion is at least 2 times greater than the ultimate tensile stress of the leaflet body.

Example 75. The method of any example herein, particularly any one of examples 70 to 74, wherein the stiff portion obtains a load at failure which is at least 2 times greater than a load at failure obtained by the leaflet body.

Example 76. The method of any example herein, particularly any one of examples 70 to 75, wherein the stiff portion is coated by a reinforcing layer.

Example 77. The method of any example herein, particularly any one of examples 65 to 76, wherein extending the legs radially through the leaflet comprising sandwiching the leaflet between the backspan and the strut.

Example 78. The method of any example herein, particularly any one of examples 65 to 77, wherein each tip comprises a slanted surface.

Example 79. The method of any example herein, particularly any one of examples 65 to 77, wherein each tip is arrow-shaped.

Example 80. The method of any example herein, particularly any one of examples 65 to 77, wherein each tip is conical.

Example 81. The method of any example herein, particularly any one of examples 65 to 78, wherein bending the legs comprises orienting the tips of each stapling member toward each other.

Example 82. The method of any example herein, particularly any one of examples 65 to 78, wherein pushing the legs toward the leaflet comprises pushing the legs toward an inner surface of the leaflet.

Example 83. The method of any example herein, particularly any one of examples 65 to 82, wherein bending the legs over the struts comprises bending the legs over an outer surface of the strut.

Example 84. The method of any example herein, particularly any one of examples 65 to 83, wherein each leg comprises, in the second bent configuration, a radial portion extending from the first bend to the second bend along a radial portion length, and a clinch portion extending between the second bend and the tip along a clinch portion length.

Example 85. The method of any example herein, particularly example 84, wherein the radial portion length is at least as great as the combined thicknesses of the leaflet and the strut.

Example 86. The method of any example herein, particularly example 84 or 85, wherein the radial portions are parallel to each other.

Example 87. The method of any example herein, particularly any one of examples 84 to 86, wherein the legs of each stapling portion are aligned with each other.

Example 88. The method of any example herein, particularly example 87, wherein the clinch portions of the stapling portion are coaxial.

Example 89. The method of any example herein, particularly example 87 or 88, wherein the backspan defines a backspan width, such that the backspan width is at least two times greater the clinch portion length.

Example 90. The method of any example herein, particularly example 87 or 88, wherein the strut defines a strut width, and wherein the strut width is at least two times greater the clinch portion length.

Example 91. The method of any example herein, particularly any one of examples 84 to 86, wherein the legs of the stapling portion are offset from each other.

Example 92. The method of any example herein, particularly example 91, wherein the clinch portions of the stapling portion are parallel to each other.

Example 93. The method of any example herein, particularly example 91 or 92, the backspan defines a backspan width, such that the clinch portion length is greater than half the backspan width.

Example 94. The method of any example herein, particularly example 91 or 92, wherein the strut defines a strut width, and wherein the clinch portion length is greater than half the strut width.

Example 95. The method of any example herein, particularly any one of examples 65 to 94, wherein providing the stapling member in a first bent configuration comprises:
providing the stapling member in a flattened configuration; and
transitioning the stapling member to the first bent configuration by bending the legs at the first bends.

Example 96. The method of any example herein, particularly example 95, wherein bending the legs at the first bends comprises bending the legs at right angles.

Example 97. The method of any example herein, particularly example 95 or 97, wherein the legs of each stapling member are coplanar with the intermediate portion and the spine in the flattened configuration.

Example 98. The method of any example herein, particularly any one of examples 95 to 97, wherein providing the stapling member in a flattened configuration comprises cutting the stapling member from a plate.

Example 99. The method of any example herein, particularly any one of examples 95 to 97, wherein providing the stapling member in a flattened configuration comprises bending a wire from which the stapling member is made to form the flattened configuration.

Example 100. The method of any example herein, particularly example 99, wherein bending the wire to form the flattened configuration comprises bending the wire to form each of the stapling portions.

Example 101. The method of any example herein, particularly example 100, wherein bending the wire to form each stapling portion comprises:
laterally bending the wire relative to the stapling member axis, forming a first planar bent extension terminating at a first tip;
extending a second planar bent extension of the wire from the first tip toward a second tip, wherein the first tip and the second tip are positioned at opposite sides of the stapling member axis; and
extending a third planar bent extension from the second tip toward the stapling member axis.

Example 102. The method of any example herein, particularly example 101, further comprising, after forming a first planar bent extension and prior to extending the second planar bent extension, bending the wire relative to the first planar bent extension to form a first lateral bent extension, and bending the first lateral bent extension to form the second planar bent extension extending therefrom.

Example 103. The method of any example herein, particularly example 102, further comprising, after extending the second planar bent extension and prior to extending the third planar bent extension, bending the wire relative to the second planar bent extension to form a second lateral bent extension, and bending the second lateral bent extension to form the third planar bent extension extending therefrom.

Example 104. The method of any example herein, particularly example 101, wherein extending a second planar bent extension comprises bending the wire to as to form an acute angle between the first planar bent extension and the second planar bent extension.

Example 105. The method of any example herein, particularly example 104, wherein extending a third planar bent extension comprises bending the wire to as to form an acute angle between the second planar bent extension and the third planar bent extension.

Example 106. The method of any example herein, particularly any one of examples 65 to 105, wherein the stapling member axis is curved.

Example 107. The method of any example herein, particularly any one of examples 65 to 106, wherein the stapling member is formed of a plastically deformable material.

Example 108. A prosthetic valve comprising:
a frame movable between a radially compressed state and a radially expanded state, the frame comprising a plurality of intersecting struts, the plurality of intersecting struts comprising recessed struts and unrecessed struts;
a valvular structure coupled to the frame and comprising a plurality of leaflets configured to regulate flow through the prosthetic valve; and
at least one compressible insert disposed within a recess formed in at least one of the recessed struts;
wherein at least one of the plurality of leaflets is coupled to at least one of the recessed struts by having a cusp end portion thereof clamped inside the recess of the corresponding recessed strut by the corresponding at least one compressible insert.

Example 109. The prosthetic valve of any example herein, particularly example 108, wherein the recess comprises an opening formed at an inner surface of the recessed strut, the opening defining an opening width.

Example 110. The prosthetic valve of any example herein, particularly example 109, wherein the recess defines a maximal recess width in a direction parallel to the opening width, and wherein the opening width is smaller than the recess width.

Example 111. The prosthetic valve of any example herein, particularly example 110, wherein the compressible insert defines an insert diameter in a free state thereof, which is compressible when pushed through the recess opening, and strives to resiliently return to the insert diameter of its free state when positioned within the recess.

Example 112. The prosthetic valve of any example herein, particularly example 111, wherein the insert diameter in the free state is greater than the opening width.

Example 113. The prosthetic valve of any example herein, particularly example 111 or 112, wherein the insert diameter in the free state is greater than a gap defined between layers of the leaflet extending through the opening.

Example 114. The prosthetic valve of any example herein, particularly any one of examples 110 to 113, wherein the recessed strut defines a strut width which is greater than the maximal recess width.

Example 115. The prosthetic valve of any example herein, particularly example 114, wherein the unrecessed struts are devoid of recesses.

Example 116. The prosthetic valve of any example herein, particularly example 115, wherein a strut width defined by at least one of the unrecessed struts is smaller than the strut width of the recessed strut.

Example 117. The prosthetic valve of any example herein, particularly any one of examples 109 to 116, wherein the recess defines a recess depth, and wherein the recessed strut defines a strut thickness which is greater than the recess depth.

Example 118. The prosthetic valve of any example herein, particularly any one of examples 108 to 117, wherein the cusp end portion is at least partially wrapped around the compressible insert within the recess.

Example 119. The prosthetic valve of any example herein, particularly any one of examples 108 to 118, wherein the valvular structure is coupled, by the at least one compressible insert, to a plurality of recessed struts that collectively form a scalloped-shaped line.

Example 120. The prosthetic valve of any example herein, particularly example 119, wherein the at least one compressible insert comprises a plurality of compressible inserts, corresponding in number to the plurality of recesses formed in the plurality of recessed struts.

Example 121. A method of assembling a prosthetic valve, comprising:
approximating a cusp end portion of at least one leaflet to an inner surface of a recessed strut of a frame of a prosthetic valve; and
pushing a compressible insert, along with a portion of the cusp end portion, into a recess formed in the corresponding recessed strut, thereby coupling the cusp end portion to the frame.

Example 122. The method of any example herein, particularly example 121, wherein pushing a compressible insert comprises pushing the compressible insert through an opening of the recess, wherein the opening defines an opening width at the inner surface of the strut.

Example 123. The method of any example herein, particularly example 122, wherein the recess defines a maximal recess width in a direction parallel to the opening width, and wherein the opening width is smaller than the recess width.

Example 124. The method of any example herein, particularly example 121 or 122, wherein pushing the compressible insert comprises squeezing the compressible insert through the opening.

Example 125. The method of any example herein, particularly any one of examples 122 to 124, wherein the diameter of the compressible insert, in a free state thereof, is smaller than the opening width.

Example 126. The method of any example herein, particularly any one of examples 122 to 125, wherein the diameter of the compressible insert, when fully disposed within the recess, is greater than a gap formed between layers of the leaflet extending through the opening.

Example 127. The method of any example herein, particularly example 123, wherein the recessed strut defines a strut width which is greater than the maximal recess width.

Example 128. The method of any example herein, particularly example 127, wherein the frame further comprises a plurality of unrecessed struts which are devoid of recesses.

Example 129. The method of any example herein, particularly example 128, wherein a strut width defined by at least one of the unrecessed struts is smaller than the strut width of the recessed strut.

Example 130. The method of any example herein, particularly any one of examples 121 to 129, wherein the recess defines a recess depth, and wherein the recessed strut defines a strut thickness which is greater than the recess depth.

Example 131. The method of any example herein, particularly any one of examples 121 to 130, wherein pushing a compressible insert comprises wrapping at least a portion of the cusp end portion around the compressible insert, such that the wrapped portion is sandwiched between the compressible insert and an inner wall of the recess.

Example 132. A prosthetic valve comprising:
a frame comprising a base portion and a plurality of vertically oriented commissure posts extending from the base portion;
a valvular structure coupled to the frame and comprising a plurality of leaflets configured to regulate flow through the prosthetic valve; and
at least one gripping tube comprising:
   an inner channel; and
   a slot extending between free ends of side arms of the gripping tube;
wherein at least one of the plurality of leaflets is coupled to the frame by the at least one gripping tube disposed around a cusp end portion of the leaflet which is at least partially wrapped around at least a part of the base portion retained within the inner channel.

Example 133. The prosthetic valve of any example herein, particularly example 132, wherein the slot defines a slot width in a free state of the slot, and wherein the side arms are configured to expand the slot when the gripping tube is pushed around the base portion, and strive to resiliently return towards each other to the slot width of the free state when no components disposed within the slot prevent them from doing so.

Example 134. The prosthetic valve of any example herein, particularly example 133, wherein the frame has a circular cross-section defining a frame tube diameter.

Example 135. The prosthetic valve of any example herein, particularly example 134, wherein the frame tube diameter is greater than the slot width.

Example 136. The prosthetic valve of any example herein, particularly any one of examples 133 to 135, wherein the leaflet is partially wrapped around the base portion of the frame, such that a cusp edge of the leaflet is disposed within the inner channel.

Example 137. The prosthetic valve of any example herein, particularly example 136, wherein the slot width is smaller than a thickness of the leaflet.

Example 138. The prosthetic valve of any example herein, particularly any one of examples 133 to 135, wherein the leaflet extends into the inner channel through the slot, is wrapped around the base portion of the frame inside the inner channel, and extends back out of the inner channel through the slot, such that a cusp edge of the leaflet is disposed out of the gripping tube.

Example 139. The prosthetic valve of any example herein, particularly example 138, wherein a thickness defined by the leaflet is greater than half the slot width.

Example 140. The prosthetic valve of any example herein, particularly any one of examples 132 to 139, wherein the base portion comprises a plurality of cusp portions disposed between the vertically oriented commissure posts, and wherein the leaflet is coupled, by the at least one gripping tube, to at least one of the cusp portions of the frame.

Example 141. The prosthetic valve of any example herein, particularly example 140, wherein the plurality of leaflets comprises three leaflets, wherein the plurality of cusp portions of the frame comprises three cusp portions, and wherein the plurality of vertically oriented commissure posts comprises three vertically oriented commissure posts.

Example 142. The prosthetic valve of any example herein, particularly example 141, wherein each leaflet of the three leaflets is coupled to a corresponding one of the three cusp portions.

Example 143. The prosthetic valve of any example herein, particularly example 142, wherein the at least one gripping tube comprises three gripping tubes, each of which coupling a corresponding one of the leaflets to a corresponding one of the cusp portions.

Example 144. The prosthetic valve of any example herein, particularly any one of examples 142 or 143, wherein each two of the three leaflets are further coupled to a corresponding one of the three vertically oriented commissure posts.

Example 145. The prosthetic valve of any example herein, particularly example 144, wherein the at least one gripping tube comprises a single gripping tube that comprises three gripping tube cusp portions, configured to couple the leaflets to the cusp portions of the frame, and three gripping tube commissure portions, configured to couple the leaflets to the vertically oriented commissure posts.

Example 146. The prosthetic valve of any example herein, particularly example 145, wherein the single gripping tube mimics the shape of the frame.

Example 147. The prosthetic valve of any example herein, particularly any one of examples 144 to 146, wherein each of the vertically oriented commissure posts is U-shaped.

Example 148. The prosthetic valve of any example herein, particularly any one of examples 132 to 147, further comprising a skirt coupled to the frame.

Example 149. The prosthetic valve of any example herein, particularly example 148, further comprising a sewing ring coupled to the skirt.

Example 150. A method of assembling a prosthetic valve, comprising:
positioning a cusp end portion of at least one leaflet between a base portion of a frame of a prosthetic valve and at least one gripping tube; and
pushing the gripping tube toward the cusp end portion and the base portion, such that a slot of the gripping tube expands to let the base portion and the cusp end portion slide into an inner channel of the gripping tube, until the base portion resides within the inner channel with the cusp end portion at least partially wrapped around the base portion, at which point side arms defining the slot of the gripping tube are allowed to snap back toward each and clamp against a portion of the leaflet extending through the slot, so as to couple the at least one leaflet to the base portion.

Example 151. The method of any example herein, particularly example 150, wherein the slot defines a slot width in a free state of the slot, prior to pushing the gripping tube.

Example 152. The method of any example herein, particularly example 151, wherein the frame has a circular cross-section defining a frame tube diameter.

Example 153. The method of any example herein, particularly example 152, wherein the frame tube diameter is greater than the slot width.

Example 154. The method of any example herein, particularly any one of examples 151 to 153, wherein pushing the gripping tube until the base portion resides within the inner channel with the cusp end portion at least partially wrapped around the base portion, comprises partially wrapping the cusp end portion around the base portion, such that a cusp edge of the leaflet is disposed within the inner channel.

Example 155. The method of any example herein, particularly example 154, wherein the slot width is smaller than a thickness of the leaflet.

Example 156. The method of any example herein, particularly any one of examples 151 to 153, wherein pushing the gripping tube until the base portion resides within the inner channel with the cusp end portion at least partially wrapped around the base portion, comprises extending the leaflet into the inner channel through the slot, wrapping the cusp end portions around the base portion inside the inner channel, and extending the leaflet back out of the inner channel through the slot, such that a cusp edge of the leaflet is disposed out of the gripping tube.

Example 157. The method of any example herein, particularly example 156, wherein a thickness defined by the leaflet is greater than half the slot width.

Example 158. The method of any example herein, particularly any one of examples 150 to 157, wherein the base portion comprises a plurality of cusp portions disposed between vertically oriented commissure posts of the frame.

Example 159. The method of any example herein, particularly example 158, wherein pushing the gripping tube toward the base portion to couple the at least one leaflet to the base portion, comprises pushing the gripping tube towards at least one of the cusp portions to couple the at least one leaflet to the at least one of the cusp portions.

Example 160. The method of any example herein, particularly any one of examples 150 to 159, further comprising, after pushing the gripping tube to couple the at least one leaflet to the base portion, suturing a skirt around the frame so as to cover the frame by the skirt.

Example 161. The method of any example herein, particularly 160, further comprising, after suturing the skirt, stitching a sewing ring to the skirt.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a frame movable between a radially compressed state and a radially expanded state;
a valvular structure coupled to the frame and comprising a plurality of leaflets configured to regulate flow through the prosthetic valve; and
at least one stapling member coupling at least one of the plurality of leaflets to the frame, the at least one stapling member comprising:
a spine extending along a stapling member axis; and
a plurality of stapling portions separated from each other by intermediate portions of the spine, wherein each stapling portion comprises at least one leg extending from a backspan;
wherein the at least one leg of each stapling portion extends through a corresponding one of the plurality of leaflets, and is bent around a corresponding strut of a plurality of struts of the frame, so as to couple the leaflet to the frame.

2. The prosthetic valve of claim 1, wherein the leaflet is sandwiched between the spine and the frame.

3. The prosthetic valve of claim 1 wherein each leg of the stapling portion is bent at a first bend relative to the backspan.

4. The prosthetic valve of claim 3, wherein the at least one leg is bent at a second bend that divides the leg to a radial portion extending between the first bend and the second bend, and a clinch portion extending from the second bend.

5. The prosthetic valve of claim 1, wherein the at least one leg of the stapling portion comprises a single leg.

6. The prosthetic valve of claim 1, wherein the at least one leg of the stapling portion comprises a pair of legs, and wherein tips of the pair of legs are oriented towards each other.

7. The prosthetic valve of claim 6, wherein one leg of the pair of legs is shorter than the other leg of the pair of legs.

8. The prosthetic valve of claim 1, wherein the stapling member is formed from a plate cut to form a planar configuration of the stapling member prior to being used for coupling the leaflet to the frame, and bent to form a second bent configuration of the stapling member when used to couple the leaflet to the frame.

9. The prosthetic valve of claim 1, wherein the stapling member is formed from a wire, and wherein the intermediate portions are intermediate wire portions.

10. The prosthetic valve of claim 9, wherein the wire is bent to form the plurality of stapling portions in a planar configuration of the stapling member prior to being used for coupling the leaflet to the frame, wherein each stapling portion comprises, in the planar configuration of the stapling member:
a first planar bent extension, terminating at a first tip;
a second planar bent extension, extending from the first tip to an opposite second tip; and
a third planar bent extension extending from the second tip.

11. The prosthetic valve of claim 1, wherein each cusp end portion comprises a stiff portion which is stiffer than a leaflet body of the leaflet.

12. The prosthetic valve of claim 1, wherein the stapling member axis is curved.

13. The prosthetic valve of claim 1, wherein the spine is zig-zagged.

14. A method of assembling a prosthetic valve, comprising:
providing a stapling member in a first bent configuration, comprising a spine extending along a stapling member axis and a plurality of U-shaped stapling portions separated from each other by intermediate portions of the spine, each stapling portion comprising a backspan and a pair of legs bent relative to the backspan at first bends of the legs;
approximating a leaflet to a strut of a frame of a prosthetic valve;
pushing the legs toward the leaflet such that tips of the legs pierce through the leaflet;
extending the legs radially through the leaflet and across the strut; and
transitioning the stapling member to a second bent configuration by bending the legs at second bends thereof over the struts, so as to couple the leaflet to the strut.

15. The method of claim 14, wherein the legs are parallel to each other at the first bent configuration.

16. The method of claim 14, wherein approximating a leaflet to the strut comprises approximating a stiff portion of the leaflet to the strut, and wherein the leaflet comprises a leaflet body extending between the stiff portion and a free edge of the leaflet.

17. The method of claim 14, wherein extending the legs radially through the leaflet comprising sandwiching the leaflet between the backspan and the strut.

18. The method of claim 14, wherein providing the stapling member in a first bent configuration comprises:
providing the stapling member in a flattened configuration; and
transitioning the stapling member to the first bent configuration by bending the legs at the first bends.

19. The method of claim 18, wherein providing the stapling member in a flattened configuration comprises cutting the stapling member from a plate.

20. The method of claim 18, wherein providing the stapling member in a flattened configuration comprises bending a wire from which the stapling member is made to form the flattened configuration.
